(19) United States
(12) Patent Application Publication
(10) Pub. No.: US 2005/0191639 A1
(43) Pub. Date: Sep. 1, 2005

(54) Method to isolate genes involved in aging

(75) Inventors: Roland Henry Contreras, Merelbeke (BE); Cuiying Chen, Balegem (BE)

Correspondence Address: TRASK BRITT, P.O. BOX 2550, SALT LAKE CITY, UT, 84110, US

(73) Assignee: Vlaams Interuniversitair Instituut Voor Biotechnologie VZW, Zwijnaarde (BE)

(21) Appl. No.: 10/852,705

(22) Filed: May 24, 2004

Related U.S. Application Data
(63) Continuation of application No. PCT/EP02/13549, filed on Nov. 28, 2002.

(30) Foreign Application Priority Data
Nov. 29, 2001 (EP) 01204600.9

(51) Int. Cl. C12Q 1/68; C12N 1/18; C12N 15/74

(52) U.S. Cl. 435/6; 435/483; 435/254.2

(57) ABSTRACT

Described is a method for isolating genes involved in aging and/or oxidative stress, by mutation or transformation of a yeast cell, subsequent screening of the mutant or transformed cells that are affected in aging and isolation of the affected gene or genes, and the use of these genes to modulate aging and aging-associated diseases in a eukaryotic cell and/or organism.

### CROSS-REFERENCE TO RELATED APPLICATIONS

This application is a continuation of International Application Number PCT/EP02/13549, filed Nov. 28, 2002, International Publication Number WO 03/045988 A2, published in English on Jun. 5, 2003, the contents of the entirety of which is incorporated by this reference.

### TECHNICAL FIELD

The present invention relates generally biotechnology, and, more particularly, to a method of isolating genes involved in aging and/or aging-associated diseases and/or oxidative stress by mutation or transformation of a yeast cell, subsequent screening of the mutant or transformed cells that are affected in aging and isolation of the affected gene or genes, and the use of these genes to modulate aging and aging-associated diseases in a eukaryotic cell and/or organism.

### BACKGROUND

Aging is a process in which all individuals of a species undergo a progressive decline in vitality leading to aging-associated diseases (AADs) and ultimately to death. The process of aging is influenced by many factors, including metabolic capacity, stress resistance, genetic stability, and gene regulation (Jazwinski, 1996). The final life span of an organism is also affected by the sum of deleterious changes and counteracting repair and maintenance mechanisms (Johnson et al., 1999).

Several approaches have been used to study aging. These include the identification of key genes and pathways important in aging, the study of genetic heritable diseases associated with aging, physiological experiment, and advanced molecular biology studies of model organisms. Among these organisms, Caenorhabditis elegans, Drosophila melanogaster and the budding yeast Saccharomyces cerevisiae have a life span that can be influenced by single gene mutations or overexpression of a particular protein (Johnson et al., 1999). Especially S. cerevisiae has been used as one of the model organisms to study the aging process (Gershon and Gershon, 2000). Yeast life span is defined as the number of daughter cells produced by mother cells before they stop dividing. This yeast cell divides asymmetrically, giving rise to a larger mother cell and a smaller daughter cell, leaving a circular bud scar on the mother cell's surface at the site of division. Thus, the age (counted in generations) of a mother cell can simply be determined by counting the number of bud scars on its surface. However, counting of the bud scars is labor intensive and time consuming and cannot be used as such as a screening method to isolate cells with an increased life span. Methods to isolate mutant yeasts with an increased life span have, amongst others, been described in PCT International Publication No. WO 95/05459 and U.S. Pat. No. 5,874,210. The latter patent describes a method to isolate a mutation which increases the number of divisions of yeast cells, comprising the labeling of the cell surface of the yeast cell with a fluorescent marker, thereby generating fluorescent yeast cells, culturing the yeast cells under conditions for growth of yeast cells for a period of time greater than the chronological life span of the strain, selecting the fluorescent cells by fluorescence-activated cell sorting and replating the fluorescent yeast cells. However, although this method may indeed give an enrichment of strains that survive longer, there is no direct selection for strains with an increased number of divisions. Non-dividing or slower dividing cells that also survive may be selected too.

### DISCLOSURE OF THE INVENTION

Disclosed herein is a method for specific isolation of old yeast mother cells, with an increased number of divisions by staining the bud-scar chitin with fluorescein isothiocyanate (FITC)-wheat germ agglutinin (WGA) lectin and sorting by a FACS apparatus, after initial enrichment of the mother cells through magnetic-based sorting. The process is presented in FIG. 1. The method can be used to isolate genes or mutations involved in aging.

Much attention has been focused on the hypothesis that oxidative damage plays an important role in aging (Shan et al., 2001; Hamilton et al., 2001) and a generally accepted relation exists between oxidative stress and aging (Tanaka et al., 2001). Moreover, mutations in genes related to protection against oxidative stress have a clear influence on life span, both in S. cerevisiae and Caenorhabitis elegans (Laun et al., 2001; Ishii, 2001). The method disclosed herein is also suitable as an indirect selection for genes involved in oxidative stress. This is especially useful in cases where screening of libraries in an endogenous system is difficult or impossible, such as the screening of mammalian or plant libraries. Screening of such libraries may lead to new genes involved in protection against oxidative stress in general, but also, in the case of mammalian cells, to genes involved in AADs and/or diseases caused by oxidative stress, especially neurodegenerative diseases such as Alzheimer's disease, Parkinson's disease, and Huntington's disease (Calabrese et al., 2001).

A frequently practiced strategy in searching genes responsible for aging is by selecting survivals after the exposure of cells to stresses. Then, because of the complexity of the process, the question constantly remaining is whether the genes picked up are in response to the stress treatment rather than involved in aging. The invention described herein, however, provides an alternative that allows direct hunting of genes with potential anti-aging functions from various libraries or library combinations of eukaryotic organisms. Yeast lines are selected in a more natural condition, in addition to advantages of high throughput, high efficiency, and short time investment. The invention has great potential for rational drug design and development of therapies and prevention in the field of age-related diseases.

It is a first aspect of the invention to provide a method to screen genes involved in aging and/or AADs and/or oxidative stress, comprising a) mutation or transformation of a yeast cell, b) cultivation of the cell, c) enrichment of the population for mother cells, d) labeling the mother cells with a WGA-based label, and e) isolation of the highly labeled cells.

To obtain a sufficient distinction between old cells and young cells, it is essential to use a marking of the bud scars that is sufficiently linear with the number of scars and is not or only weakly interacting with other cell wand compounds. Surprisingly, we found that WGA can bind with the chitin in the bud scar, without major interference with other cell compounds, so that the amount of WGA bound is a reliable measurement of the number of bud scars. The WGA bound is then measured using a WGA-based label. A WGA-based label, as used herein, may be any kind of label that allows quantifying the amount of WGA bound to the cell and may be, as a non-limiting example, WGA coupled to a stain or a detectable antibody that binds to WGA. Detectable antibodies are known to the person skilled in the art and may be, as a non-limiting example, rabbit antibodies that can be detected by a labeled anti-rabbit antibody. The labeling of mother cells with a WGA-based label may be a one-step process, wherein labeled WGA is bound to the cell, or a two-step process, wherein in a first step, WGA is bound to the bud scars and in a second step, the bound WGA is labeled. A preferred embodiment is a method according to the invention, wherein the WGA-based label is FITC-labeled WGA.

Preferably, the isolation of highly stained cells is based on FACS sorting. Methods for the enrichment of the population of mother cells are known to the person skilled in the art and may be based on, as a non-limiting example, staining of the cell wall of the cells at a certain point in the growth phase followed by continuation of the culturing and sorting of the stained cells. Alternatively, the cells may be antibody labeled.

Preferably, the enrichment of the population of mother cells is a magnetic-based sorting. Instead of being based on a global cell wall labeling as described above, the enrichment of the population of mother cells may be based on the labeling of a fraction of the mother cells, such as a bud-scar-based labeling. In fact, the enrichment of the mother cells may be carried out by a first WGA-based labeling and sorting, wherein the enriched mother cells are subjected to a second WGA-based labeling and sorting. The labeling method in the first and second round may be different.

### BRIEF DESCRIPTION OF THE FIGURES

FIG. 1: Scheme of the bud-scar sorting (BSS) system for yeast M-cells. The BBS system contains two major steps. The first step is shown at the left side of the figure and depicts magnetic sorting of biotinylated M-cells and regrowth of sorted M-cells to desired generations when needed. The second step is shown at the right side of the figure and depicts WGA staining of bud scars and sorting of longer life M-cells according to bud-scar staining.

FIG. 2: Flow cytometric assay of yeast cells labeled with WGA-FITC and streptavidin-PE. Yeast cells (M-cells) are grown for five to six generations (G5-6) after biotin labeling, sorted via MACS, and then simultaneously labeled with WGA-FITC and streptavidin-PE. Panel A shows a clear separation of the PE red-fluorescent mother cells (gated M-cell) from the non-PE fluorescent daughter cells (gated D-cells). Panel B hardly detects the PE fluorescent signal in the depleted daughter cells. Panels C and D depict the layout of FSC versus SSC, the gated M-cells mainly appearing at higher FSC/SSC values representing a large cell size population (C) compared to a small cell size population of D-cells at lower FSC/SSC values (D). Panels E and F indicate that the M-cell population gives strong WGA-FITC staining (E) than the D-cell population (F).

FIG. 3: Bud-scar staining of yeast cells. INVSc-1 cells (M-cells) were biotinylated and cultured in SD medium. M-cells at G5-6 were magnetically sorted. Staining of bud scars with WGA-FITC was revealed with a Zeiss LSM410 confocal microscope.

FIG. 4: Screen of a human cDNA library via FACS. A cDNA library from HepG2 hepatoma cells was transformed into the yeast strain INVSc-1 (pEX2) (see Materials and Methods). The transformed yeast population was first labeled with biotin and then cultured in S-glycerol medium. The initial biotinylated M-cells of approximately G14 (14 generations) were obtained by running two magnetic sorting and regrowth cycles and were then double labeled with WGA-FITC and streptavidin-PE. The older mother cells were gated according to PE staining and big cell size which represented as high FSC (A). Flow sorted older mother cells (gate Old-M) show a strong WGA-FITC signal (B).

FIG. 5: Flow cytometric dead cell assay using PI staining. Flow cytometric analysis of cell death using PI staining was performed in a ferritin L chain clone (pEX2-FL) and its parent line of INVSc-1 (pEX2). Yeast cells were grown up to six generations. The gate R1 was set around PI-positive cells that cover the dead cells, the gate R2 around the PE-positive cells that represents the M-cell population and the gate R3 around the D-cell population. In panel A, it shows 16.3% dead cells for the ferritin L chain clone. In panel B, a 33% dead cell was observed in the control line.

FIG. 6: Resistance of ferritin-containing yeast to H₂O₂ (IniM) stress. Cells transformed with the plasmids as indicated were exponentially grown at 30° C. to an OD₆₀₀ of approximately 0.5. Cells were treated with 1 mM H₂O₂ during various times. Samples were diluted and plated on YPD solid media to monitor cell viability. C12-ferritin indicates the cell line containing the ferritin-fragment expression vector of pGAL10-FL. Its parent line transformed with the empty vector of pSCGAL10-SN was used as control.

FIG. 7: Life span of C. elegans carrying the human Ferritin Light Chain (FTL) gene. Animals were injected with a L4759 plasmid containing human FTL gene. Controls were injected with empty plasmids. pRF4 containing the dominant phenotypic marker rol-6 (su 1006) was coinjected in both cases. Results are cumulative from four independent experiments with more than 25 animals per trial. “Life span” is defined as the day when the first transformed larvae hatched until their death. Animals carrying copies of the human FTL gene lived significantly longer (13.54±0.269 days) than controls (12.50±0.266 days).

FIG. 8: Study of the aging phenotype of yeast Δfob1 strain by the mixed-growth system. A mixture of Δfob1 strain and parent BY4742, were biotinylated and grown in SD medium as described in example 7. G20 (the point after 20 generations) was obtained by running three cycles of magnetic sorting and regrowth. The results show an increased frequency of Δfob1 cycling M-cells at G20, illustrating a longer life span.

FIG. 9: Comparison of the viability of FTL strain with its parents. The initial mixture of M-cells (FTL and INVSc-1) was biotinylated and grown in minimal SD and S-glycerol media as described in materials and methods. The ratio of viable M-cells in the mixture at different ages was determined by plating. Data for cells grown in the FTL gene inducing S-glycerol medium are presented at the right side of the figure, while data for the control are shown on the left side, indicating that the difference in aging is clearly due to the ferritin expression. In a separate experiment, doubling times of both strains were carefully tested and found to be equal.

FIG. 10: Ferritin L prevents fast aging in presence of iron in yeast as tested by micromanipulator experiment. Life spans of human partial ferritin and full ferritin transformed in strain BY4741. S-raffinose was used as carbon source for inducing expression of ferritin. An excess of iron was added in the medium with 500 μM FAC and 80 μM ferrichrome. At least 60 cells were included in each of three life span assays. Both partial and full ferritin had a longer average life span (17.85 G and 15.58G) than the control (12.19).

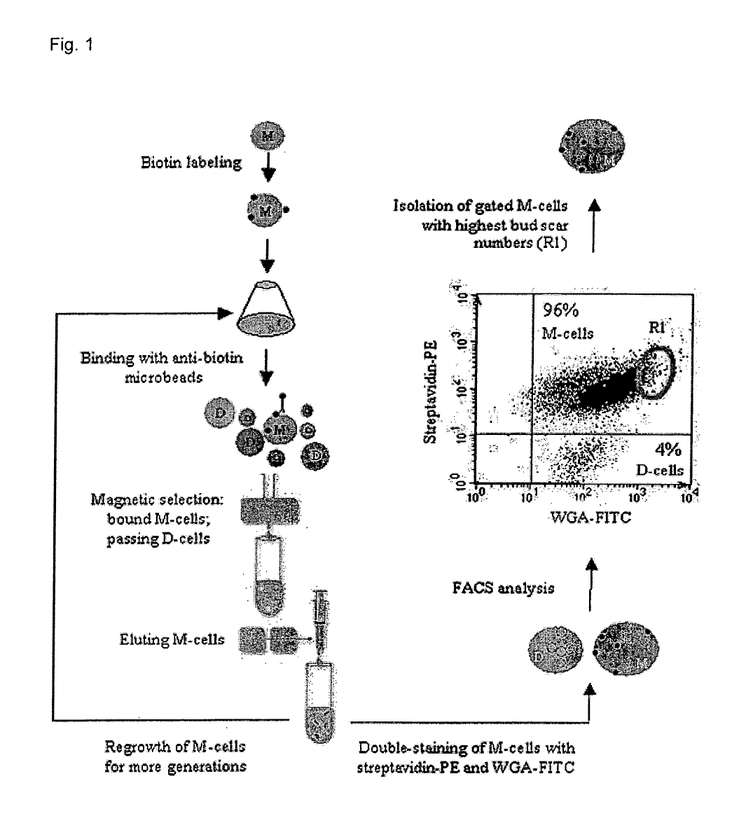

### DETAILED DESCRIPTION OF THE INVENTION

Methods to mutate yeasts are known to the person skilled in the art and include, but are not limited to, chemical and physical mutagenesis, such as ethyl methane sulphonate (EMS) treatment or UV treatment. Methods to transform yeast are also known to the person skilled in the art and include, but are not limited to, protoplast transformation, lithium acetate-based transformation and electroporation. The yeast transformation may be carried with one or more nucleic acids, up to a complete library. The nucleic acid used is not necessarily yeast nucleic acid, but may be from any origin, as long as it is functionally expressed in yeast. Preferred examples of nucleic acids are mammalian nucleic acids, such as human nucleic acid, and plant nucleic acid, wherein the nucleic acids are cloned in a yeast expression vector. Preferably, the yeast is transformed with an expression library. The nucleic acid that is transcribed into mRNA does not necessarily translate into protein, but may exert its effect as antisense RNA. Indeed, it is an additional advantage of the method that it can detect in one screening experiment both the effect of overexpression of a protein, as well as the effect of down-regulation of a protein by blocking the translation of an endogenous messenger by a homologous antisense RNA resulting from the expression library.

Another aspect of the invention is a gene or functional gene fragment isolated with the method of the present invention. The functional fragment may encode for a polypeptide that directly affects aging and/or an AAD and/or oxidative stress, or it may be transcribed into antisense RNA, which affect aging and/or an AAD and/or oxidative stress by silencing an endogenous gene. Preferably, the gene or functional gene fragment is selected from the nucleic acid listed in Table 2. More preferably, the gene or functional gene fragment comprises a sequence as represented in SEQ ID NOS:1, 3, 5, 7, 8, 9, 11, 13, 15, 16, 17, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53. Even more preferably, the gene or gene fragment consists essentially of one of the foregoing sequences. Even more preferably, the gene or functional gene fragment consists of one of the foregoing sequences.

A preferred embodiment is a gene fragment, isolated with the method of the present invention, consisting essentially of SEQ ID NO:11, preferably consisting of SEQ ID NO:11. Another preferred embodiment is a gene fragment, isolated with the method of the present invention, consisting essentially of SEQ ID NO:16, preferably consisting of SEQ ID NO:16.

Still another aspect of the invention is the use of a gene or functional gene fragment isolated with the method according to the invention to modulate aging and/or to modulate the development of AADs and/or to protect against oxidative stress. Preferably, the modulation is an inhibition of aging. Preferably, the gene or gene fragment is selected from the nucleic acids listed in Table 2. More preferably, the gene or gene fragment comprises a sequence as represented in SEQ ID NOS:1, 3, 5, 7, 8, 9, 11, 13, 15, 16, 17, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53. Even more preferably, the gene or gene fragment consists essentially of one of the foregoing sequences. Even more preferably, the gene or gene fragment consists of one of the foregoing sequences.

A preferred embodiment uses a functional gene fragment consisting essentially of SEQ ID NO:11, preferably consisting of SEQ ID NO:11. Another preferred embodiment is the use of a gene fragment, isolated with the method, consisting essentially of SEQ ID NO:16, preferably consisting of SEQ ID NO:16.

Another aspect of the invention is a polypeptide encoded by a gene or functional gene fragment isolated with a method according to the invention. Preferably, the modulation is an inhibition of aging and/or inhibition of the development of an AAD. Preferably, the polypeptide is enclosed by a nucleic acid listed in Table 2. More preferably, the polypeptide is encoded by a nucleic acid comprising SEQ ID NO:1, 3, 5, 7, 8, 9, 11, 13, 15, 16, 17, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53. Even more preferably, the polypeptide is encoded by a nucleic acid consisting essentially of one of the foregoing sequences. Even more preferably, the polypeptide is encoded by a nucleic acid consisting of one of the foregoing sequences. Even more preferably, the polypeptide comprises SEQ ID NOs:2, 4, 6, 10, 12, 14, 18, or 20. Even more preferably, the polypeptide consists essentially of SEQ ID NOS:2, 4, 6, 10, 12, 14, 18 or 20. Even more preferably, the polypeptide consists of SEQ ID NOS:2, 4, 6, 10, 12, 14, 18 or 20.

A preferred embodiment is a polypeptide essentially consisting of SEQ ID NO:12, preferably consisting of SEQ ID NO:12. Still another preferred embodiment is a polypeptide encoded by a nucleic acid essentially consisting of SEQ ID NO:16, preferably consisting of SEQ ID NO:16.

Still another aspect of the invention is the use of a polypeptide encoded by a gene or functional gene fragment isolated with a method according to the invention to modulate aging and/or to modulate the development of an AAD and/or to protect against oxidative stress. Preferably, the modulation is an inhibition of aging and/or inhibitor of the development of an AAD. Preferably, the polypeptide is encoded by a nucleic acid selected from the nucleic acids listed in Table 2. More preferably, the polypeptide is encoded by a nucleic acid sequence selected from the group consisting of SEQ ID NOS:1, 3, 5, 7, 8, 9, 11, 13, 15, 16, 17, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, and 53. More preferably, the polypeptide comprises SEQ ID NO:2, 4, 6, 10, 12, 14, 18 or 20. Even more preferably, the polypeptide consists essentially of SEQ ID NO:2, 4, 6, 10, 12, 14, 18 or 20. Most preferably, the polypeptide consists of SEQ ID NO:2, 4, 6, 10, 12, 14, or 20. A preferred embodiment is the use of a polypeptide essentially consisting of SEQ ID NO:12, preferably consisting of SEQ ID NO:12, to modulate aging and/or to modulate the development the development of an AAD. Preferably, the modulation is an inhibition of aging and/or an inhibition of the development of an AAD. Still another preferred embodiment is the use of a polypeptide encoded by a nucleic acid comprising SEQ ID NO:16, preferably consisting essentially of SEQ ID NO:16, more preferably consisting of SEQ ID NO:16, to modulate aging and/or to modulate the development the development of an AAD.

Still another aspect of the invention is the use of an antisense RNA encoded by a gene or a functional gene fragment, isolated with a method according to the invention, to modulate aging and/or to modulate the development of an AAD. In such an application, the gene or functional gene fragment is operationally linked to a promoter in such a way that an antisense RNA, complementary to the mRNA encoding the polypeptide normally encoded by the gene or gene fragment, is transcribed. Preferably, the gene or functional gene fragment encoding the antisense RNA comprises SEQ ID NO:7, 8, or 15. Even more preferably, the modulation of aging is an inhibition of aging and/or an inhibition of the development the development of an AAD.

Definitions

“Gene” as used herein refers to a region of DNA that is transcribed into RNA and subsequently preferentially, but not necessarily, translated into a polypeptide. The term is not limited to the coding sequence. The term refers to any nucleic acid comprising the region, with or without the exon sequences, and includes, but is not limited to, genomic DNA, cDNA and messenger RNA. As, on the basis of these sequences, it is evident for the person skilled in the art to isolate the promoter region, the term “gene” may include the promoter region when it refers to genomic DNA.

“Nucleic acid” as used herein refers to a polymeric form of nucleotides of any length, either ribonucleotides or deoxyribonucleotides. This term refers only to the primary structure of the molecule. Thus, this term includes double- and single-stranded DNA and RNA. It also includes known types of modifications, for example, methylation, “caps” substitution of one or more of the naturally occurring nucleotides with an analog.

“Functional fragment of a gene” involved in aging is every fragment that, when tested with the method according to the invention, still gives a positive response. Typically, functional fragments are fragments that have deletions in the 5′ and/or 3′ untranslated regions. Alternatively, the functional fragment may be an antisense fragment encoding an RNA that is silencing an endogenous gene or functions as RNAi. As the coding sequence on its own is also considered as a functional fragment, it is evident for the person skilled in the art that it may be functional when it is placed between suitable heterologous 5′ and 3′ untranslated sequences.

“Polypeptide” refers to a polymer of amino acids and does not refer to a specific length of the molecule. This term also includes post-translational modifications of the polypeptide, such as glycosylation, phosphorylation and acetylation.

“Aging” as used herein includes all forms of aging, particularly also AADs. AADs are known to the person skilled in the art and include, but are not limited to, arteriosclerosis, Parkinson's disease and Alzheimer's disease.

The invention is further described with the aid of the following illustrative Examples.

### EXAMPLES

Materials and Methods to the Examples

Strains and Media

The following S. cerevisiae strains were used: INVSc-1 (Invitrogen, San Diego, Calif.); BY4741 and BY4742 (Euroscarf, Frankfurt, Germany), as well as the BY4742-derived Δfob1 strain (Euroscarf; Accession No. Y14044). Strains were grown at 30° C. in rich YPD medium (2% dextrose, 2% bactopeptone and 1% yeast extract) or minimal SD medium (0.67% yeast nitrogen base without amino acids, 2% dextrose and 0.077% complete supplement mixture—uracil). The INVSc-1 and BY4741 strains used for library screening were grown in S-glycerol, S-galactose or S-raffinose media, where dextrose is replaced with 3% glycerol, 2% galactose or 2% raffinose, respectively. S-glycerol was used to induce expression of genes cloned in pEX2, whereas S-galactose was used to induce expression of genes cloned in pSCGAL10-SN. Media were solidified with 2% agar.

Cloning and Overexpression of a Human cDNA Library

To recover mRNA from various responses, a pool of equal proportions of human HEPG2 cells, subjected to different treatments, was used for library construction. These treatments included heat shock for 1.5 hours at 42.5° C., 1 mM dithiothreitol, 100 U/ml interleukin-6 and 10⁻⁷ M dexamethasone. Construction of cDNA libraries was carried out essentially as described previously (Declercq et al. 2000). cDNA was cloned at the site of SfiI/NotI in the vectors pEX2 (BCCM/LMBP Plasmid Collection, Ghent University, Belgium; Accession No. 2890) and pSCGAL10-SN (BCCM/LMBP Plasmid Collection, Accession No. 2471). cDNA expression is driven by the cytochrome c promoter in pEX2 and by the GAL10 promoter in pSCGAL10-SN. Yeast strain INVSc-1 was used as the host for pEX2 library transformation. The pSCGAL10-SN library was transformed to the BY4741 strain. Transformations were performed as described previously (Gietz and Woods, 2001). Approximately 3.5×10⁵ colonies from each transformation were produced.

Magnetic Sorter-Based Preparation of Yeast Mother Cells (M-cell)

Cells were cultured at 30° C. in liquid medium, such as minimal SD medium or in the specific induction medium, to OD₆₀₀ of 0.7-1 and were collected by centrifugation. All cells harvested were used as M-cells. The biotin labeling of M-cells was carried out essentially as described previously (Smeal et al., 1996). Before labeling, M-cells were washed twice with cold phosphate-buffered saline (PBS; pH 8.0), resuspended in PBS to a concentration of 2.5×10⁷ cells/ml and then incubated with 0.1 mg/ml Sulfo-NHS-LC-Biotin (Pierce Chemical Company, Rockford, Ill.) for 30 minutes at room temperature under gentle shaking. The free biotin reagent was removed by two washings with PBS. Biotinylated M-cells were grown in liquid medium for a desired number of generations (up to G7 in our conditions; culture was not allowed to exceed OD₆₀₀=1).

The separation of mother cells from the daughter cells they produced was carried out via magnetic cell sorting. This was realized by coupling the biotinylated mother cells to magnet beads by incubating 10⁷ mother cells with 80 μl of Anti-Biotin MicroBead (Miltenyi Biotec, Germany) in 1 ml PBS pH 7.2 for 1 hour at 4° C. Unbound beads were removed by washing twice with PBS. M-cells were isolated with a magnetic sorter according to the supplier's protocol (Miltenyi Biotec). When needed, these sorted M-cells can be further grown in liquid medium for additional generations and isolated again by the magnetic sorting system.

The purity of sorted mother cells was determined on the basis of streptavidin binding. About 10⁷ biotinylated cells were stained with 3 μg streptavidin-conjugated R-phychoerthrin (PE) (Molecular Probes) in 1 ml of PBS pH 7.2 for 1 hour at room temperature in total darkness. Then cells were washed twice with PBS and suspended in 2 ml of PBS pH 7.2. The yeast cells with more bud scars were recognized as a high intensity of FITC signals.

WGA-Based Bud-Scar Staining

The bud scars of yeast cells were stained with fluorescein isothiocyanate (FITC)-labeled WGA lectin (Sigma). The staining was carried out by adding 10⁷ yeast cells together with 12 μg WGA-FITC in 1 ml of PBS pH 7.2 for 1.5 hours in the dark at room temperature. After two washing steps with PBS to remove the free WGA-FITC reagent, yeast cells were resuspended with PBS to a concentration of 0.5×10⁷ cell/ml for FACS analysis.

Propidium Iodide (PI) Staining

PI (Sigma) was freshly dissolved in PBS buffer to a final concentration of 1 mg/ml as stock solution. For staining, yeast cells were suspended in PBS pH 7.2 to approximately 10⁷ cell/ml and then, 3 μl of PI stock solution was added into 1 ml yeast cell suspension. The sample was run within five to ten minutes on a flow cytometer (Becton Dickinson), which is capable of measuring red fluorescence (with a band pass filter>650). No washing steps were included.

Set-Up of Becton Dickinson FACScan

Analysis of FITC, PE and PI labeling of the cell population was accomplished at an excitation wavelength of 488 nm, using a 15 mWatt argon ion laser. FITC emission was measured as a green signal (530 nm peak fluorescence) by the FL1 detector, PE was measured as an orange signal (575 nm peak fluorescence) by the FL2 detector, and PI was measured as a red signal (670 nm peak fluorescence) by the FL3 detector. The FACScan flow cytometer (Becton Dickinson) was operated according to the standard protocol of the supplier. For multi-color staining, electronic compensation was used among the fluorescence channels to remove residual spectral overlap. A minimum of 10,000 events was collected on each sample. Analysis of the multivariate data was performed with CELLQuest software (Becton Dickinson Immunocytometry System).

Transformation and Aging Assay in Nematode

The expression vector of human ferritin fragment (FTL) for C. elegans was derived from L4759 by replacing the GFP with FTL fragment.

Wild-type C. elegans strain (N2) was used as host for FTL expression. The animals were cultured and handled as described (Brenner, 1974). The transient overexpression of human FTL was carried out according to Jin (1999) using an Eppendorf FemtoJet-TransferMan NK injection system (Eppendorf, Leuven, Belgium). Twenty-five to thirty worms were injected with plasmid carrying the human FTL gene or control plasmid. Plasmid pRF4, which carries the dominant rol-6 (su1006) allele was coinjected to mark transformed progeny. After a one-hour recovery period in M9 buffer, injected animals were allowed to lay eggs for approximately 40 hours on plates containing nematode growth medium (NGM) and a lawn of E. coli bacteria (OP50) as food. Transformed eggs were predominantly laid during the last 20 hours resulting in a fairly synchronous experimental cohort. Subsequently, the injected animals were removed and progeny (F1) was allowed to grow at 24° C. Fourth stage larvae or young adults showing the Roller phenotype were transferred onto separate plates (NGM+OP50) containing 300 μM 5-fluoro-2′-deoxyuridine (FUDR, Sigma) to prevent progeny (F2) production. Live/dead scoring was carried out daily. Lifespan is defined as the day when the first transformed larvae hatched until their death.

Construction of a Full Ferritin Clone

A ferritin PCR fragment (end to stop cordon) was generated from the hepatoma cDNA library by using specific primers (5′-ctacgagcgtctcctgaagatgc-3′ (SEQ ID NO:54) and 5′-cgcggatccaagtcgctgggctcagaaggctc-3′ (SEQ ID NO:55)). This fragment was cloned directly into the TOPO vector (Invitrogen, The Netherlands) and then digested with NotI, generating a NotI fragment. Subsequently, the NotI fragment was inserted in the NotI site of ferritin light fragment clone (PGAL 10-FL), resulting a 750 bp full ferritin clone in pSCGal-SN-10.

### Example 1

### Magnetic-Based Sorting of Yeast M-Cells

To use yeast as an aging model, the first step needed is the development of a system, which allows the isolation of a relatively pure population of old yeast cells. The method for distinguishing and separation of S. cerevisiae cells between generations is based on the fact that daughter cells have a wall that is newly formed and do not have any detectable wall remnants of the mother cells. Cells from an overnight culture of S. cerevisiae strain INVSc-1 in minimal SD medium were covalently coated with biotin and designated as mother cells (M-cells). The M-cells were inoculated into fresh medium and allowed to grow for five to six generations as determined by the cell density that is measured by a UV-visible spectrophotometer (Shimadzu). After loading with anti-biotin beads, M-cells were sorted out using a magnetic sorter or MACS (Materials and Methods).

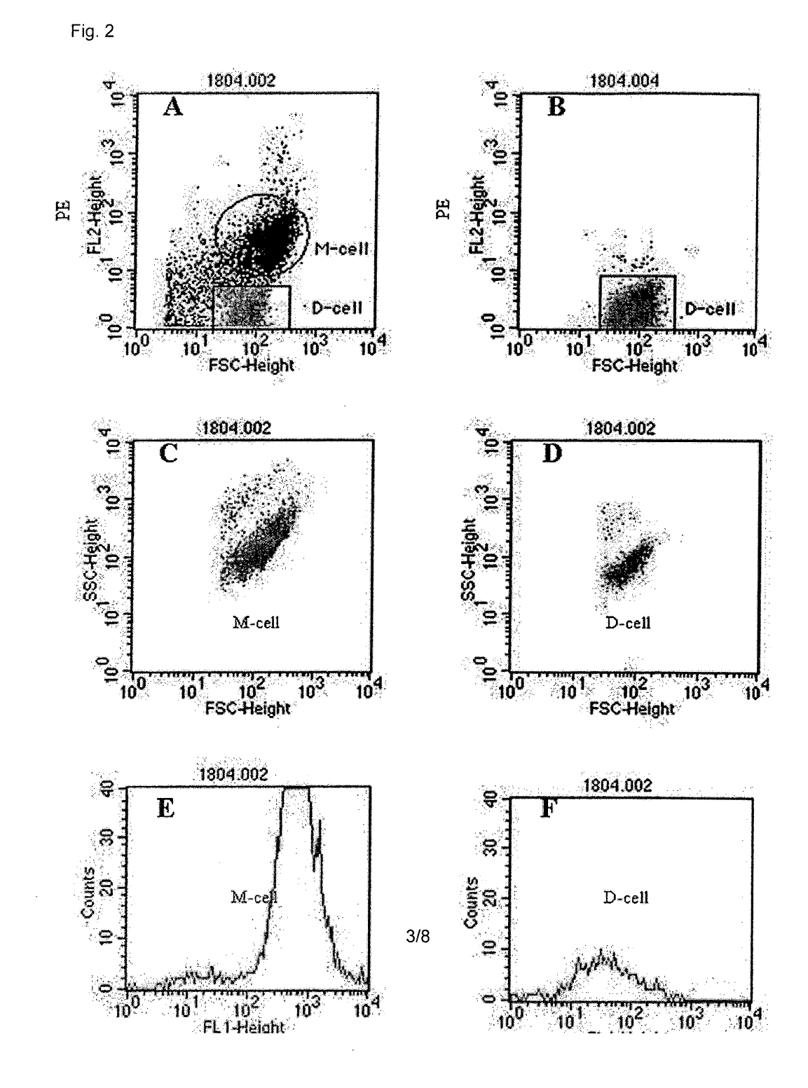

The purity of the collected M-cells was determined by staining with streptavidin-PE, which specifically binds to biotin coated on the cell wall of M-cells, followed by flow cytometric analysis. Due to the reaction of biotin with streptavidin-PE, high-density staining of biotinylated M-cells was shown. As shown in FIG. 2A, there was clear separation between stained M-cells and unstained daughter cells (D-cells) populations. Gate and marker were positioned to exclude D-cells from the M-cell population. In the layout of FSC versus SSC, as the matter of fact, the gated M-cells mainly appeared at high FSC/SSC values representing a large cell size population (FIG. 2C) compared to a small cell population of D-cells which mainly located at lower FSC/SSC values (FIG. 2D). Statistic analysis showed that the purity of the isolated M-cells reached more than 85%. FIG. 2B shows a PE staining performed on a depleted D-cell population, which hardly shows any positive signal.

### Example 2

### WGA-Based Staining for Analysis of Yeast Life Span

Wheat germ agglutinin (WGA, Triticum vulgare) is the first lectin of which the amino acid sequence was completely determined (Wright, 1984). WGA is a mixture of several isolectins (Rice and Etzler, 1975). Sharing similar carbohydrate binding properties with other lectins, WGA reacts strongly with the chitobiose core of asparagines-linked oligosaccharides, especially with the Manβ(1,4)GlcNAcβ(1,4)GlcNAc trisaccharide (Yamamoto et al., 1981).

One of the most striking features of the cell surface during aging, S. cerevisiae is the accumulation of chitin-containing bud scars. To verify whether WGA can be used for specific labeling of chitin in yeast bud scars, the yeast strain INVSc-1 (pEX2) was incubated with the FITC-conjugated WGA. The enriched, magnetically sorted M-cells were subjected to WGA reaction.

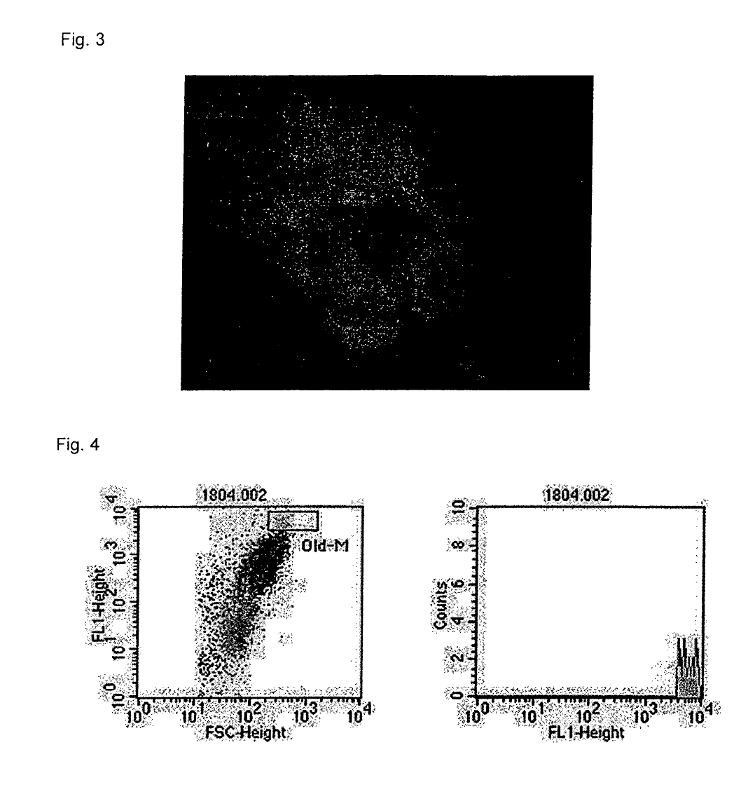

Under a fluorescence microscope, it was found that the major part of the fluorescent signal for WGA-FITC staining was co-localizing with the bud scar rings (FIG. 3). Moreover, the number of stained bud scars (six bud scars) was consistent with the expected age of the M-cells as estimated by cell density measurement of the culture (five to six generations). This observation demonstrated that, under the conditions used, WGA is specifically binding to the chitin of bud scars and hardly gives any fluorescence caused by binding to compounds in the normal cell wall. Therefore, the possibility was examined to use WGA as a tool to stain bud scars for analysis of yeast life span. The isolated M-cells and depleted D-cells (as seen in FIGS. 2C and 2D) were simultaneously stained with streptavidin-PE and WGA-FITC. As shown in FIGS. 2E-2F, D-cells that were negative for streptavidin-PE staining showed low FITC signal (FIG. 2F), whereas M-cells, which were positive in streptavidin-PE staining, showed a much stronger FITC staining (FIG. 2E). Under the fluorescent microscope, it was observed that most M-cells contained five to six bud scar rings, which were strongly labeled by WGA-FITC, while most D-cells had only one to two bud scar rings. This observation indicated that there was a good linear correlation between the number of bud scars and the intensity of fluorescence. Therefore, it was assumed that WGA could be used as a tool for bud scar-specific staining in budding yeast cells.

### Example 3

### Application of Using WGA to Screen a Human cDNA Library

It has been reported that overexpression of certain human genes in yeast might have an influence in the frequency distribution of the yeast population (Gershon and Gershon, 2000). This overexpression of a single gene, which modulates the longevity in a single-cell system, has opened up the field of aging study to the power of yeast genetics. To screen human genes that might be involved in aging processes, a cDNA library from hepatoma cells was constructed and transferred into the yeast strain INVSc-1 (pEX2) (see Materials and Methods). The transformed yeast population was first labeled with biotin and then cultured in a Bioreactor (AppliTek), for about 14 generations, as deduced from the cell density. According to the method described above, the initial biotinylated M-cells were isolated by magnetic beads described herein and then labeled with WGA-FITC. By flow cytometric analysis (FIG. 4A), the M-cell population had a high density of WGA-FITC staining (gate M-cell), whereas D-cells showed a lower fluorescent staining (gate D-cell). As shown in FIG. 4, older M-cells, gated as Old-M population that were supposed to have a longer life span, were marked on high FITC intensity combined with high FSC, and then were flow sorted by FACS. From nine colonies, the gene, overexpressed in the yeast cell was sequenced and the results are summarized in Table 1. The growth rate was tested by measuring the doubling time of each strain in the liquid medium. The result showed that the growth rate of all nine clones, as well as the parent line, were similar.

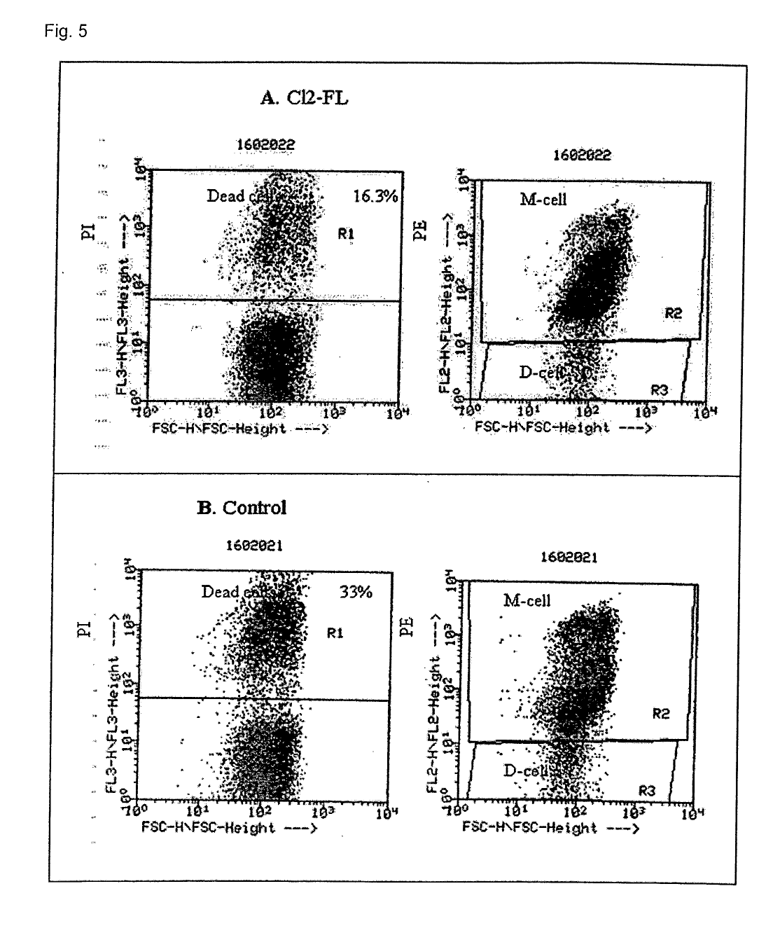

One of the colonies contained a gene fragment encoding ferritin light (FL) chain (M1147.1; Afl 19897.1). To verify whether the overexpression of this gene could influence the life-span of the yeast cell or not, an analysis of cell death using PI staining was performed in this ferritin L chain clone (CI2-FL) using its parent line of INVSc-1 (pEX2) as a control. Ten million M-cells for each cell line were isolated. As shown in FIG. 5, on the FSC versus PE (FL2) dot plot, a gate R2 was set around the PE-positive cells that represent the M-cell population, while a gate R3 was set around the D-cell population. At the same time, on the FSC versus PI (FL3) dot plot, a gate R1 was set around PI-positive cells that cover the dead cells. As seen in FIG. 5, cell death in culture occurred mainly in the M-cell population but was barely detected in the D-cell population. Statistical analysis for dead cells (PI-positive) showed a higher frequency in control cells (33% death) compared to that in CI2-FL cells (16.3% death). This result indicates that overexpression of the human ferritin L chain in yeast cells prevents early cell death.

### Example 4

### Additional Screening Experiments

To confirm the usefulness of the method, additional screening experiments were set up using the same outline as described above, both using the pEX2 library and the pSCGAL10-SN library. The results of the additional screening experiments are listed in Table 2 and identified by their GenBank® accession number. Several results of the first screening have been confirmed, illustrating the usefulness and the reliability of the method.

### Example 5

### Protective Effect of the Ferritin Fragment on Hydrogen Peroxide Treatment

One of the colonies contained a gene fragment encoding ferritin light (FTL) chain (M1147.1; Afl19897.1) cloned in pSCGAL10-SN. The plasmid was indicated as pGAL10-FL. Ferritin is ubiquitously distributed in the animal kingdom. It is composed of two subunits, the heavy chain (H) and the light chain (L). Ferritin plays a major role in the regulation of intracellular iron storage and homeostasis. One of the functions is to limit iron availability for participation in reactions that produce free oxygen radicals, which have the potential to damage lipids, proteins and DNA. Indeed, several reports have implicated that ferritin is involved in the protection against oxidative stress, such as stress induced by hydrogen peroxide. However, there is not such ferritin-like protein present in yeast and anti-oxidative activity of ferritin fragments was never demonstrated. To test whether the human ferritin fragment plays a role as an antioxidant in yeast, we examined the partial-ferritin L clone (CI2-ferritin), which was isolated by the method according to the invention, against H₂O₂ stress.

The condition for treatment of the cells was essentially the same as described by Jamieson et al. (1994). Exponential phase cultures of strain BY4741 that contained the empty vector pSCGAL10-SN (Control) and the ferritin expression vector (FTL indicated as CI2-ferritin), respectively, were grown aerobically in S-galactose medium at 30° C. The cell cultures were then challenged to a lethal concentration of H₂O₂ (1 mM). Cell survival was monitored by taking samples at 0, 30 and 60 minutes, diluting the samples in the same medium and plating aliquots on YPD plates.

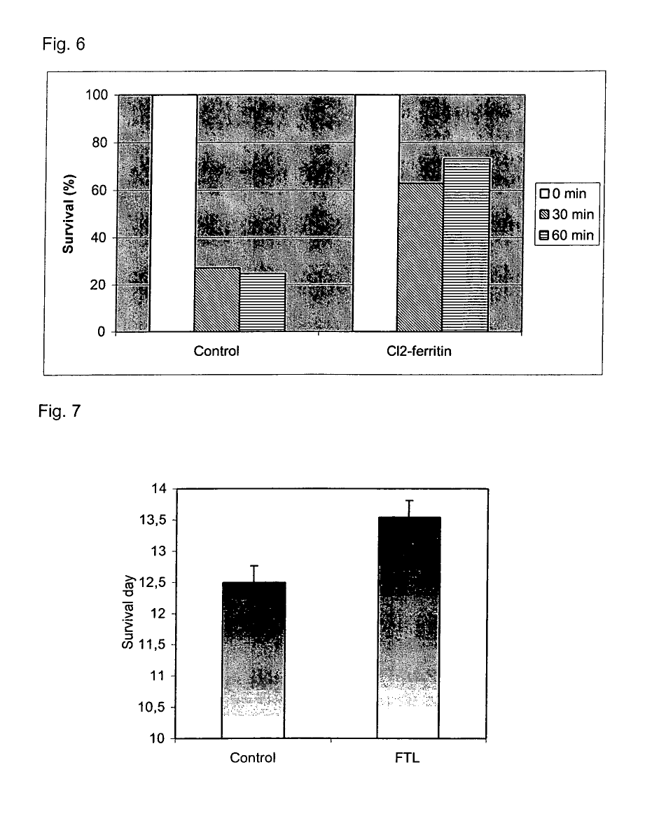

The experiment showed that, compared with the control line, ferritin cells are significantly more resistant to treatment with 1 mM H₂O₂ (FIG. 6).

### Example 6

### Transgenic Nematode Overexpressing the Ferritin Light Chain

Although on the cellular level there might be some conserved mechanism of aging processes throughout evolution (Martin et al., 1996), it is easy to imagine that in different species some underlying distinctive ways of intercellular regulation also contribute to reach their fate (Guarente 2001). In this sense, results from other organisms may provide a closer vision on the postulated function of the human FTL gene involved in aging. Therefore, it was tested whether FTL might affect lifespan in C. elegans, a multicellular organism, too. Indeed, as shown in FIG. 5, animals carrying human FTL genes appeared to have an average life of 13.5 days, which is 8% longer than the control line and statistically significant (p=0.006, two-way ANOVA). Many reports in C. elegans, Drosophila and mice are consistent with the hypothesis that oxidative damage accelerates aging and that increased resistance to oxidative damage can extend lifespan (Finkel and Holbrook, 2000). The consistency that the expression/overexpression of human FTL gene was in favor in extending the life span in mono-cellular yeast and multi-cellular nematode supports the postulation that ferritin extends life span in cells, probably by protecting cells from oxidative stress, in a wide range of species.

A frequently practiced strategy in searching genes responsible for aging is by selecting survivals after exposure of cells to stresses. The question constantly exists that the genes picked up might be in response to the stress treatment rather than involved in aging because of the complicity of the process. The screening method described here, however, provides an alternative that allows direct hunting of genes with potential anti-aging functions from various libraries or library combinations of eukaryotes. Yeast lines are selected in a more native condition and also with advantages of high throughput, high efficiency, and short time consumption. The invention has a great potential in application in rational drug design and therapies development in the field of age-related diseases preventing/treatments.

### Example 7

### Elaboration of the Mixed Culture Experiments

Based on the fact that a parental yeast strain and its direct derivative have a similar cell cycle rate, a mixed culture method has been developed to verify the long-living character of a transformed yeast strain when these strains are grown together in the same culture.

Two (or possibly more than two) yeast strains with a similar growth rate are initially mixed in the same culture in an equal ration (50% each in the case of two strains). The strains can be distinguished from each other by the use of a selective marker. The initial inoculated cells, called mother cells (M-cells), are labeled with biotin and are grown together in the same culture during their entire life span. Mother cells at different generation points are sampled and collected by a magnetic system (MACS) similar to the method described in Example 1. The ratio of living M-cells from the two strains is determined by the use of the selective marker. If the two strains have a similar life span, the ratio of two viable strains will stay the same at different generation time points; otherwise, the ratio will change. This method is essentially based on the screening method, wherein the identification of the long-living cells is not carried out by WGA staining but by direct count of the number of living mother cells of the transformed stain(s), compared to the number of living mother cells of the parental strain.

FOB1 is required for the replication fork block. An FOB1 mutation results in a decreased rDNA recombination rate and an increase in yeast life span of 70%. The growth rate of the Δfob1 mutant strain, as measured, is similar to its parental strain. Therefore, the long-living Δfob1 strain with its parental strain BY4742 was used to develop the mixed-growth system.

The initial mother cells were prepared as follows: a first pre-culture was made by inoculating BY4742 and Δfob1 cells (from freshly grown on a SD plate) in 5 ml of SD medium, respectively. The culture was incubated at 30° C. on a shaker at 250-300 rpm overnight. A second pre-culture was made by inoculating the first pre-culture into 5 ml of SD medium at a cell density of OD₆₀₀=0.001˜0.005. These cells were incubated until the culture reached a cell density of OD₆₀₀=0.5˜0.7. Cells were collected by centrifugation of the culture at 4° C. for five minutes at 3000 rpm. The cell pellet was washed twice with pre-cooled PBS (pH 8) and resuspended in PBS at a cell density of OD₆₀₀=5 (approximately 5×10⁷ cells/m.). The biotinylation of cells was performed in an Eppendorf tube, in 1 ml reaction volume consisting of 0.5 ml of the above-mentioned cells (2.5×10⁷ cells) and 0.5 ml of 1 mg/ml biotin (Sulfo-NHS-LC-Biotin). The mixture was incubated for 30 minutes at room temperature with a gentle shaking. The biotinylated cells were centrifuged for 5 minutes at 13000 rpm and washed twice with 1 ml of cold PBS to get rid of free biotin. These cells were used as initial mother cells (M-cells).

A 100 ml mixed-growth culture of BY4742 and Δfob1 was set up by inoculating 1×10⁷ biotinylated M-cells from each strain (mother cells) at the ratio of 1:1 in a SD medium. The mixed-growth culture was incubated at 30° C. on a shaker at 250-300 rpm. The culture density was not allowed to exceed OD₆₀₀>1.

After growing several generations (up to seven generations in this condition), the M-cells were labeled with anti-biotin microbeads and isolated using the magnetic system (MACS). The purity of M-cells was determined by FACS (fluorescence-activated cell sorter) after staining M-cells with streptavidin conjugated with FE. Using these conditions, more than 90% M-cells could be obtained. After the final magnetic sorting, the ratio of viable M-cells was measured.

Mixed M-cell samples were plated at about 500 cells per plate on YPD and YPD/geneticin plates to determine the ratio of mother cells of the two strains at different generation points. Plates were incubated for three days at 30° C. The ratio of BY4742 and Δfob1 mother cells was monitored by counting the colonies on the two kinds of plates. The total viable number of M-cells could be determined on the YPD plate, while the number of viable Δfob1 M-cells could be derived from the YPD/geneticin plate.

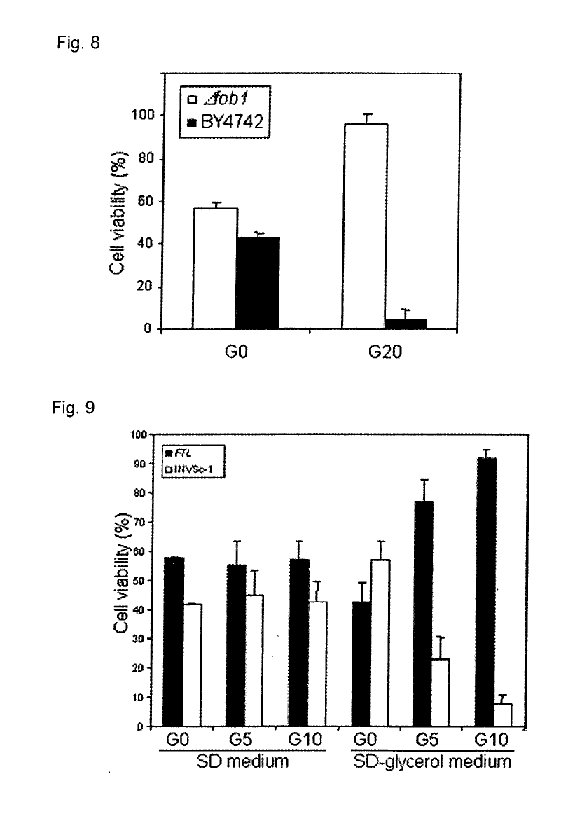

As shown in FIG. 8, the mixed M-cell group had similar amounts of the two strains at G0, while at G20, M-cells from Δfob1 were dominant (96%) among the cells sorted and collected with the magnetic sorting system. This result confirms that the mixed-growth method could indeed be used to distinguish the longer living yeast strain from its control.

### Example 8

### Confirmation of Aging Phenotype of Ferritin Strain by Mixed-Growth System

A kinetic analysis for growth rate of the ferritin yeast (FTL) and its parental strain INVSc-1 (with a geneticin-selectable marker) revealed a similar rate. About an equal amount of two strains was mixed, as described above, but using S-glycerol medium to obtain induction of the ferritin expression. This mixed culture was subjected to a mixed-growth experiment for determining their life span differences. After examination of the longevity of a mixed-growth of these two cell types by the mixed-growth system and subsequent plating, we found that the ferritin line was predominant in the viable M-cell group after a growth of ten generations (FIG. 9). Growth of a mixture of these two lines in SD medium, in which the expression of ferritin was not induced, revealed a constant viable FTL/INVSc-1 ratio. This indicates that the extended longevity of the FTL strain, compared to the age-matched INVSc-1 strain, is caused by the expression of human FTL.

### Example 9

### Independent Confirmation of Effect on Life Span by Ferritin

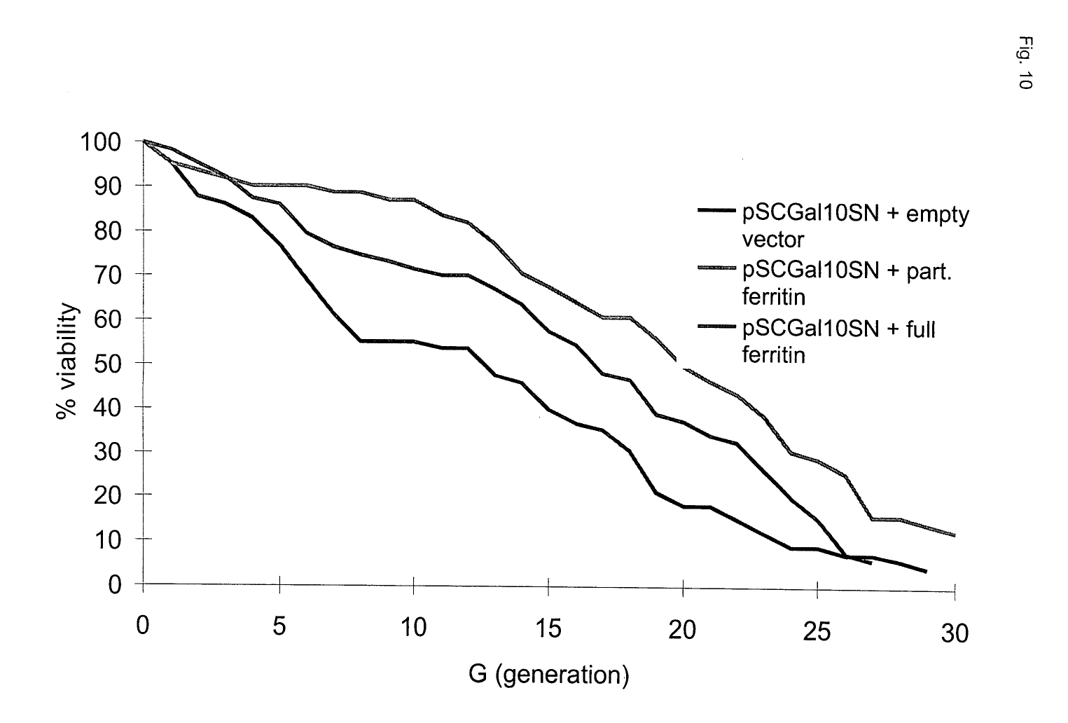

Iron is an essential nutrient for virtually every organism because it is required as an essential cofactor for many proteins. However, excess iron can generate, via the Fenton reaction, highly toxic-free radicals generating oxidative damage to the cell. Thus, cellular iron concentration must be tightly controlled. To exam whether expression of human ferritin in yeast could protect cell death upon excess iron, the life span analysis of ferritin strains was carried out by micromanipulator as described previously (Kennedy et al., 1994) with the following slight modifications. Cells were pre-grown on non-inducing SD medium (2% glucose), shifted to inducing S-raffinose (2% raffinose) medium with 500 μM ferric ammonium citrate (FAC) and 80 μM ferrichrome (Sigma), and grown for at least two generations. Cells were taken from this logarithmically growing liquid culture and transferred at low density on S-raffinose with 500 μM FAC and 80 μM ferrichrome plate (2% agar). The cells were then incubated at 30° C. overnight. Virgin daughter cells were isolated as buds from populations by micromanipulator and used as the starting mother cells for life span analysis. For each successive bud removed from these mother cells, they were counted one generation older. Cells were grown at 30° C. during the day and at cold room overnight. Each experiment includes at least 60 cells. The statistical analysis of life span was carried out by a Wilcoxin's test. The life span of full ferritin and partial ferritin yeast strains were significantly extended by 10 to 15% compared to their parent strain BY4741 (FIG. 10). This result confirms that the human ferritin light chain prevents fast aging in presence of iron in yeast.

TABLE 1
results of the screening of 9 positive clones

| | Insert length | | Identification | |
|---|---|---|---|---|
| Clone number | (approx.) | | (Based on homology) | SEQ ID NO: |
| 1 | 1.6 | kb | Humanin | 1 |
| 2 | 883 | bp | APOA1 | 3 |
| 3 | 1 | kb | Ribosomal protein P0 | 5 |
| 4 | 2.8 | kb | glutamyl tRNA synthetase | 7⁽¹⁾ |
| 5 | 2.4 | kb | GRSF-1 | 8⁽¹⁾ |
| 7 | 700 | bp | ALDH1 | 9 |
| 8 | 416 | bp | ferritin light chain | 11 |
| 9 | 1 | kb | Ribosomal protein S2 | 13 |
| 12 | 500 | bp | Histone H2A | 15⁽¹⁾ |

⁽¹⁾antisense

Table 2: Results of further screening experiments. The results are grouped in mitochondrial functions, ribosomal proteins, and other genes with known function, unknown functions and chromosomal fragments. The results of the first screening are not repeated in this table; however, several genes, like the ferritin fragment, have been identified in more than one screening experiment. The sequences are identified by their GenBank® accession number. The length of the isolated fragment may differ from the GenBank® sequence and is normally shorter. Where relevant, the fragment is indicated, using the nucleotide's numbers of the GenBank® sequence.

| | | | |
|---|---|---|---|
| Clone Name | Function | Accession Number | Orientation |

Mitochondrion

| | | | |
|---|---|---|---|
| 1E3/6D8 | ATP synthase 6 mRNA | AF368271 | sense |
| 2C10 | mitochondrial ATP synthase subunit 9, | U09813 | sense |
| | P3 gene copy, mRNA, nuclear gene | | |
| | encoding mitochondrial protein | | |
| 5D9 | ATP synthase, H+ transporting, | NM_001697 89-745 | sense |
| | mitochondrial F1, complex, O subunit | | |
| | (oligomycin sensitivity conferring | | |
| | protein), (ATP5O) | | |
| 9B11 | ADP/ATP translocase mRNA, 3′ end | J03591 | sense |
| 4D7/7H1/12D3/13E9 | NADH dehydrogenase 1 | BC009316 380-685; 10-684; | sense |
| | | 138-645; 10-490 | |
| 6C11 | NADH dehydrogenase 1 | BC009316 | sense |
| 7E11 | NADH dehydrogenase subunit 5 | AF339086 | sense |
| | (MTND5) mRNA, RNA 4, complete | | |
| | cds; mitochondrial gene for | | |
| | mitochondrial product | | |
| 10G3 | mitochondrion cytochrome b gene, | U09500 | sense |
| | partial cds | | |
| 12F1 | cytochrome c oxidase subunit III gene, | AF004341 | sense |
| | mitochondrial gene encoding | | |
| | mitochondrial protein, partial cds | | |
| 2A7 | ubiquinol-cytochrome c reductase core | BC003136; 763-1131 | sense |
| | protein II | | |
| 1B12 | monocyte chemotactic protein-3 | X72308 | |
| | (MCP-3) | | |
| 1F9/12H12 | Wnt-13; mitochondrial DNA | Z71621; 1-348; 12-372 | sense |
| 7C1 | 12S ribosomal RNA gene, partial | AY012136 | sense |
| | sequence; and tRNA-Val gene, | | |
| | complete sequence; mitochondrial | | |
| | genes for mitochondrial products | | |
| 11B7 | MRPS16 mRNA for mitochondrial | AB049948 | sense |
| | ribosomal protein S16 | | |
| 10F3/14H4/14H5/7B10 | clone IMAGE: 5581122, mRNA; | BC035832.1\| | sense |
| | haplotype N1b mitochondrion | \|AF381999; 228-726; | |
| | | 330-953; 134-1028; | |
| | | 2059-2658 | |

Ribosome

| | | | |
|---|---|---|---|
| 1S_3 | ribosomal protein P0 | BC005863 | sense |
| 3A5 | ribosomal protein, large, P1 | NM 001003.2 | sense |
| 12E12 | ribosomal protein L12 (RPL12) | NM_000976 | sense |
| 6F8 | ribosomal protein L14 | BC029036 | sense |
| 1D12 | ribosomal protein L31 (RPL31) | NM_000993 | sense |
| 1S_9 | ribosomal protein S2 | NM_002952 | sense |
| 6D6 | ribosomal S3 (RPS3) | NM_001005.2 | sense |
| 3D1/4G10 | ribosomal protein S3A | BC030161 | sense |
| | v-fos transformation effector | M84711 | sense |
| | protein (Fte-1) | | |
| 3B9 | ribosomal protein S4, X-linked | NM 001007 | sense |
| | (RPS4X) | | |
| | scar protein | M22146 | sense |
| 4H2 | ribosomal protein S4, Y-linked | NM 001008 | sense |
| | (RPS4Y) | | |
| 10E8 | ribosomal protein S5 | BC018151 | sense |
| 14G6 | ribosomal protein S6 (RPS6) | NM_001010.2\| | sense |
| 4C5 | ribosomal protein S10 | BC005012 | sense |
| 4B5/2A3 | ribosomal protein S11 | BC016387 | sense |
| | Mus musculus RAD21 homolog | NM_009009 | sense |
| | (S. pombe) (Rad21) | | |
| 11E4 | ribosome protein S16 | nm 001020 | sense |
| 2E6 | ribosomal protein S17 mRNA | M13932 | sense |
| 1D1 | ribosomal protein S25 | BC004986 | sense |
| 1C11 | Wilm's tumor-related protein | M64241 | sense |
| | (QM) mRNA; RPL10 | | |

Other genes from the 4^{th} screen (pEX2 library)
Unknown functions

| | | | |
|---|---|---|---|
| 2H4 | likely ortholog of mouse gene rich | NM 031299.2\|; 346-end | sense |
| | cluster, C8 | | |
| 3C2 | clone FLC0593 | AF113701 | sense |
| 4C11 | similar to putative, clone MGC: 33177 | BC028387; 1905-end | sense |
| | IMAGE: 4823662 | | |
| 4D10 | full length insert cDNA clone ZE03C06 | AF086514 | sense |
| 4E9 | hypothetical protein dJ465N24.2.1 | NM_020317; 874-1431 | sense |
| | (DJ465N24.2.1) | | |
| 6F6 | Similar to RIKEN cDNA 1110012M11 | BC007883 | sense |
| | gene | | |
| 6H8 | cDNA FLJ31039 fis, clone | AK055601; 1869-end | sense |
| | HSYRA2000221 | | |
| 7F6 | cDNA FLJ13305 fis | AK023367 | sense |
| 8C10 | hypothetical protein FLJ23018 | NM_024810 | sense |
| | (FLJ23018) | | |
| 9F4 | Similar to hypothetical protein | BC024001; 3-end | sense |
| | FLJ10751 | | |
| 9G10 | hypothetical protein BC013073 | NM_138391 | sense |
| | (LOC92703) | | |
| 10G6 | similar to C50F4.16.p (LOC256281) | XM_170755 | antisense |
| 12E6 | hypothetical protein MGC955 | NM_024097.1\| | sense |
| 14D4 | clone IMAGE; 4778940 mRNA | BC031919.1; 3-end | sense |
| 5S-15/114 | cDNA DKFZp434O159 | AL133593 | sense |
| 5S-21/57 | hypothetical protein FLJ10081 | NM_017991 | sense |
| 7F11 | cDNA FLJ38528 fis | AK095847 | sense |
| 11H3 | cDNA FLJ14279 fis | AK024341; 362-end | sense |
| 5C4 | Similar to KIAA0674 protein | BC026048 | sense |
| 2E2 | cDNA FLJ14385 fis, clone | AK027291; 3-end | sense |
| | HEMBA1002212, weakly similar to | | |
| | TYROSINE-PROTEIN KINASE 2 | | |
| 7G6 | KIAA0776 protein (KIAA0776) | NM_015323; 3-end | sense |

Chromosome DNA seq.

| | | | |
|---|---|---|---|
| 10D4 | DNA sequence from clone | AL031668; | sense |
| | RP1-64K7 on chromosome | 66383-66970 | |
| | 20q11.21-11.23 Contains the | | |
| | EIF2S2 gene for eukaryotic | | |
| | translation initiation factor 2 | | |
| | subunit 2 (beta, 38 kD), a putative | | |
| | novel gene, the gene for | | |
| | heterogeneous nuclear | | |
| | ribonucleoprotein RALY or | | |
| | autoantigen P542, an RPS2 | | |
| | (RPS4) (40 S ribosomal protein | | |
| | S2) pseudogene, ESTs, STS, | | |
| | GSSs and two CpG islands | | |
| 2A6 | PAC clone RP3-414A15 from | AC005225; | sense |
| | 14q24.3 | 93459-93782 | |
| 2D3 | DNA sequence from clone | AL451084; | antisense |
| | RP11-357H24 on | 42698-42510; with | |
| | chromosome 10 | polyA | |
| 2F8 | chromosome 17, clone | AC004231; | sense |
| | hRPC.1110_E_20 | 42223-429716; with | |
| | | polyA | |
| 3F11 | BAC clone CTD-2314H8 | AC079338; | sense |
| | | 21007-21487 | |
| 4F7 | chromosome 1 clone RP11-109I2 | AC091609; | sense |
| | | 155982-156333 | |
| 11B8 | DNA sequence from clone | AL603888 | antisense |
| | RP11-735A5 on chromosome 1 | | |
| 12A9 | chromosome 18, clone | AC106037.9\| | sense |
| | RP11-13N13 | | |

### REFERENCES

Brenner, S. (1974) The genetics of Caenorhabditis elegans. Genetics 77:71-94.
Calabrese, V., Scapagnini, G., Giuffrida Stella, A. M., Bates, T. E. and Clark, J. B. (2001) Mitochondrial involvement in brain function and dysfunction: relevance to aging, neurodegenerative disorders and longevity. Neurochem. Res. 26:739-764.
Declercq, W., Logghe, M., Fiers, W. and Contreras, R. (2000) “Cloning and expression of cytokine genes,” in the book of Cytokine Molecular Biology, Balkwill, F. R. (ed.), IRL Press, Oxford, UK; pp 1-17.
Ernst, J. F. and Chan, R. K. (1985) Characterization of Saccharomyces cerevisiae mutants supersensitive to aminoglycoside antibiotics. J. Bacteriol. 163, 8-14.
Finkel, T. and Holbrook, N. J. (2000) Oxidants, oxidative stress and the biology of aging. Nature 408:239-47.
Gershon H. and Gershon D. (2000) The budding yeast, Saccharomyces cerevisiae, as a model for aging research: a critical review. Mech. Aging Dev. 120:1-22.
Gietz, R. Daniel and Woods, Robin A. (2001) Genetic transformation of yeast. Biotechniques 30:816-20, 822-6, 828 passim.
Guarente, L. (2001) SIR2 and aging—the exception that proves the rule. Trends Genet. 17:391-2.
Hamilton, M. L., Van Remmen, H., Drake, J. A., Yang, H., Guo, Z. M., Kewitt, K., Walter, C. A. and Richardson, A. (2001) Does oxidative damage to DNA increase with age. Proc. Natl. Acad. Sci. 98:10469-10474.
Ishii, N. (2001) Oxidative stress and aging in Caenorhabitis elegans. Free Radic. Res. 33:857-864.
Jamieson, D. J., Rivers, S. L. and Stephen, D. W. (1994) Analysis of Saccharomyces cerevisiae proteins induced by peroxide and superoxide stress. Microbiology 140:3277-3283.
Jazwinski, S. M. (1996) Longevity, genes, and aging. Science 273:54-59.
Jin Y. (1999) Transformation. In C. elegans—A practical approach, Hope I. A., ed. (New York, N.Y., Oxford University Press), pp. 69-96.
Johnson, F. B., Sinclair, D. A. and Guarente, L. (1999) Molecular biology of aging. Cell 96:291-302.
Kennedy, B. K., Austriaco, N. R., and Guarente, L. (1994) Daughter cells of S. cerevisiae from old mothers display reduced life span. J. Cell Bio. 127, 1985-1993.
Laun, P., Pichova, A., Madeo, F., Fuchs, J., Ellinger, A., Kohlwein, S., Dawes, I., Frohlich, K. U. and Breitenbach, M. (2001) Aged mother cells of Saccharomyces cerevisiae show markers of oxidative stress and aging.
Martin, G. M., Austad, S. N. and Johnson, T. E. (1996) Genetic analysis of aging: role of oxidative damage and environmental stresses. Nat. Genet. 13:25-34.
Rice, R. H. and Etzler, M. E. (1975) Chemical modification and hybridization of wheat germ agglutinins. Biochemistry 14:4093-4099.
Shan, F., Nowell, T. R. Jr. and Taylor, A. (2001) Removal of oxidatively damaged proteins from lens cells by the ubiquitin-proteasome pathway. Exp. Eye Res. 73:229-238.
Smeal, T., Claus, J., Kennedy, B., Cole, F., Guarente, L. (1996) Loss of transcriptional silencing causes sterility in old mother cells of S. cerevisiae. Cell. 84:633-42.
Tanaka, T., Nakamura, H., Nishiyama, A., Hosoi, F., Masutani, H., Wada, H. and Yodoi, J. (2001) Redox regulation by thioredoxin superfamily; protection against oxidative stress and aging. Free Radic. Res. 33, 851-855.
Yamamoto, K., Tsuji, T., Matsumoto, I. and Osawa, T. (1981) Structural requirements for the binding of oligosaccharides and glycopeptides to immobilized wheat germ agglutinin. Biochemistry 20:5894-5899.
Wright, C. S. (1984) Structural comparison of the two distinct sugar binding sites in wheat germ agglutinin isolectin II. J. Mol. Biol. 178:91-104.

1. A method of screening genes in a cell involved with aging, aging associated diseases (AADs), and/or in oxidative stress, said method comprising:
a) mutating or transforming a yeast cell,
b) cultivating said yeast cell to form a population of transformed or mutated yeast cells,
c) enrichment of the population for mother cells,
d) labelling said mother cells with a wheat germ agglutinin (WGA)-based label, and
e) isolating the highly labelled mother cells, thus screening said genes.

2. The method according to claim 1, wherein said WGA-based label is fluorescein isothiocyanate (FITC)—conjugated WGA.

3. The method according to claim 1, wherein isolation is by a fluorescence activated cell sorting (FACS)—based sorting.

4. The method according to claim 2, wherein isolation is by a fluorescence activated cell sorting (FACS)—based sorting.

5. The method according to claim 1, wherein enrichment is magnetic-based sorting.

6. The method according to claim 1, wherein the yeast cell is transformed by a yeast expression library.

7. The method according to claim 6, wherein said yeast expression library expresses mammalian DNA or plant DNA.

8. A nucleotide sequence isolated by a process comprising:
a) mutating or transforming a yeast cell,
b) cultivating said yeast cell to form a population of transformed or mutated yeast cells,
c) enrichment of the population for mother cells,
d) labelling said mother cells with a wheat germ agglutinin (WGA)-based label,
e) isolating the highly labelled mother cells,
f) analyzing the highly labeled mother cells to identify nucleotide sequences.

9. The nucleotide sequence of claim 8, wherein said nucleotide sequence comprises a sequence selected from the group consisting of SEQ ID NO:1, SEQ ID NO:3, SEQ ID NO:5, SEQ ID NO:7, SEQ ID NO:8, SEQ ID NO:9, SEQ ID NO:11, SEQ ID NO:13, SEQ ID NO:15, SEQ ID NO:16, SEQ ID NO:17, SEQ ID NO:19, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, and SEQ ID NO:53.

10. A method of modulating aging and/or protecting against oxidate stress in a cell, comprising using the nucleotide sequence of claim 8 to modify said cell.

11. The method according to claim 10, wherein said nucleotide sequence comprises SEQ ID NO:11 or SEQ ID NO:16.

12. An isolated polypeptide encoded by the nucleotide sequence of claim 8.

13. A method of modulating aging and/or protecting against oxidate stress in a cell, said method comprising interacting said cell with a polypeptide encoded by the nucleotide sequence of claim 8.

14. The method according to claim 13, wherein said nucleotide sequence comprises a sequence selected from the group consisting of SEQ ID NO:2, SEQ ID NO:4, SEQ ID NO:6, SEQ ID NO:10, SEQ ID NO:14, SEQ ID NO:18, and SEQ ID NO:20.

15. The method according to claim 13, wherein said polypeptide comprises SEQ ID NO:12.

16. The method according to claim 13, wherein said polypeptide is encoded by SEQ ID NO:16.

17. An isolated peptide encoded by an isolated nucleotide sequence selected from the group of sequences consisting of SEQ ID NO:1, SEQ ID NO:3, SEQ ID NO:5, SEQ ID NO:7 through SEQ ID NO:11, SEQ ID NO:13, SEQ ID NO:15 through SEQ ID NO:17, SEQ ID NO:19, and SEQ ID NO:21through SEQ ID NO:53.

18. A method of modulating aging and/or protecting against oxidate stress in a cell, said method comprising:
interacting said cell with an isolated peptide encoded by a nucleotide sequence selected from the group of sequences consisting of SEQ ID NO:1, SEQ ID NO:3, SEQ ID NO:5, SEQ ID NO:7 through SEQ ID NO:11, SEQ ID NO:13, SEQ ID NO:15 through SEQ ID NO:17, SEQ ID NO:19, and SEQ ID NO:21 through SEQ ID NO:53.

19. A method of modulating aging and/or protecting against oxidate stress in a cell, said method comprising:
interacting said cell with an isolated peptide encoded by a nucleotide sequence selected from the group consisting of SEQ ID NO:11 and SEQ ID NO:16.

20. A method of modulating aging and/or protecting against oxidate stress in a cell, said method comprising:
interacting said cell with an isolated peptide comprising SEQ ID NO:12.


SEQUENCE LISTING (55 sequences)

SEQ ID NO: 1 - DNA, 1551 bp, Homo sapiens
  human HN1 cDNA
caaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctntttggacactaggaaaaaaccttgtagagagagtaaaaaatttaacacccatagtaggcctaaaagcagccaccaattaagaatagcgttcaagctcaacacccactacctaaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcgagtaccctaaccgtgcaaaggtagcataatcacttgttccttaattagggacctgtatgaatggctccacgagggttcagctgtctcttacttttaaccagtgaaattgacctgcccgtgaagaggcgggcatgacacagcaagacgagaagaccctatggagctttaatttattaatgcaaacagtacctaacaaacccanaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattcctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacttcaaattcctccctgtncgaaaggacaagagaaataaggcctacttcacaaagcgccttcccccgtaaatgatatcatctcaacttagtattatacccacacccacccaagaacagggtttgtt

SEQ ID NO: 2 - protein, 24 aa, Homo sapiens
  human HN1 cDNA
MAPRGFSCLLLLTSEIDLPVKRRA

SEQ ID NO: 3 - DNA, 884 bp, Homo sapiens
  human APOA1 cDNA fragment
aggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 4 - protein, 275 aa, Homo sapiens
  human APOA1 cDNA fragment
GGPPRPFRMKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 5 - DNA, 771 bp, Homo sapiens
  human ribosomal protein P O cDNA fragment
cggatatgaggcagcagtttctccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcacccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttcttt

SEQ ID NO: 6 - protein, 231 aa, Homo sapiens
  human ribosomal protein P O cDNA fragment
GSSFSKEDLTEIRDMLLANKVPAAARAGAIAPCEVTVPAQNTGLGPEKTSFFQALGITTKISRGTIEILSDVQLIKTGDKVGASEATLLNMLNISPFSFGLVTQQVFDNGSIYNPEVLDITEETLHSRFLEGVRNVASVCLQIGYPTVASVPHSIINGYKRVLALSVETDYTFPLAEKVKAFLADPSAFVAAAPVAAATTAAPAAAAAPAKVEAKEESEESDEDMGFGLFD

SEQ ID NO: 7 - DNA, 2769 bp, Homo sapiens
  human glutamyl tRNA synthase antisense cDNA
agaatttagctgttctttattgacatggaatttggggtggcgagggtagccacaccctccaggaggatgaggtaggttcagtgcttccagctcacacctttcctgggtcttccttcagtgtgacagttcggttaaagacaagctttccctgatggctgtctggatccacggagaaatatccaagacgctcaaactggaacttgtcgaagggttttgccagggccacagagcagtccactaatgctgcatccaccacgtgtagtgatgccaggttcaggtcacttaaaaatccaccaggcacctcagtaggatcttcagggttcttgtgctggaatagtcgctcatagaggcgaacctcacacatcaaaggctgtgacacccagtgaataaaggcctttggcttctctccagcatctgcccgtctgcaggtcacctccagactctctacacaaccactggggcccttgacaacatgctgcagctcaatgacgtagcctgtatgcctcaggcccacaggctggccccaagccaggcgcttaaatcctggctctggctcctccttgaagtcagtcctctcaatgaagacaatgggtgcaaagggaacctgatggaagcctttggtctcatcagctgggaagttgggcacctggatgtccaaggacttggcagcaggaaagttggtgatgatgacccgtagtgactccagcacagccatggctcgtggggctgtgtcattcagcacatcacgcacacaggcttctagaagatgtggctccattgtggtttgtgccacagtcactcccacccgggcacagaagttgttgatggcctcaggtgggaagccccgccgtcgcagggccgtgagtgtaaagagccgtgggtcatcccagtcccgcacagcaccagttgctacaagctggaggatcttcctcttagagacaacagcatagtgcaggttgaggcggccatactcccactgcacagggcaatagacgtncagtgcattgcaaagccagaagtaggaagagcgtcgggcctggaattccttggtgcanagtgagtgagtgatgtgctcgatggagtcacagaggcagtgtgtgtagtcgtaggtgggatagatgcaccatttgtcccctgtgcggtggtgtggtgtatacttgactcgataggctacagggtccatcttgccatcctccatcaccagcttcatccgtagtgtggcctcgccctctgaaaacttgcccttgcgcattgcctcaaagagcagcagtgactcctccatgggacggtctctccagggtgaaggcagagtattatggcctttgagctcctctcctcgctggtggcacacataagccagacccctgcggatgagctccacagcccacgcatatagctggtcaaaatagtcagacgcatatgtgactttgtaaggtgtgtagcctagccaggctaccatgtcacagatggccgtgaagaactttgcttcctccttctcagggttggtgtcatcaaaacgcagaaaacagatgccattgttggccttggcatagccaaagttgaaattgatggctttggcatgtccaatatgcaggattccattgggttctggcgggaaccgggtacgtacctgcccaccaggttgcttccgaacatcctgctcaccggtacaccaggggttggaaaaaccacactaggcaaagaacttgcgtcaaaatcaggactgaaatacattaatgtgggtgatttagctcgagaagtctgatcatcggatatcatggagtctggcaagacggcttctcccaagagcatgccgaaagatgcacagatgatggcacaaatcctgaaggatatggggattacagaatatgagccaagagttataaatcagatgttggagtttgccttccgatatgtgaccacaattctagatgatgcaaaaatttattcaagccatgctaagaaagctactgttgatgcagatgatgtgcgattggcaatccagtgccgcgctgatcagtcttttacctctcctcccccaagagattttttattagatattgcaaggcaaagaaatcaaacccctttgccattgatcaagccatattcaggtcctaggttgccacctgatagatactgcttaacagctccaaactataggctgaaatctttacagaaaaaggcatcaacttctgcgggaagaataacagtcccgcggttaagtgttggttcagttactagcagaccaagtactcccacactaggcacaccaaccccacagaccatgtctgtttcaactaaagtagggactcccatgtccctcacaggtcaaaggtttacagtacagatgcctacttctcagtctccagctgtaaaagcttcaattcctgcaacctcagcagttcagaatgttctgattaatccatcattaatcgggtccaaaaacattcttattaccactaatatgatgtcatcacaaaatactgccaatgaatcatcaaatgcattgaaaagaaaacgtgaagatgatgatgatgacgatgatgatgatgatgactatgataatctgtaatctagccttgctgaatgtaacatgtatacttggtcttgaattcattgtactnatattaaacatgcatgctggatgttttcaagttgtgttttagaaaactaataataatgagtaaacacagttaccatacttttcaattgaaatgaaggtttttcatcagccttaaaagtgtaagaaaaataaagttgtcattcatt

SEQ ID NO: 8 - DNA, 2233 bp, Homo sapiens
  human G rich sequence factor antisense cDNA
tgcacataaagttagtttattaatgactatattttgaagccagccattttgtccaatatttaaataacaagctgtttaatattaaagcagaaagtactgccacattgtgacagaagtacagctttatccataaacccttcacacaattatacattaaatgctatttttatttaagcaaggcacccctacttgttctaaaatatgggatgtactactccatttaaaaagcaaatgaggagactgatttttttcctatctagagctggtttaaattcaagtgaagccattaattttcttaccagtctggactgttctgacatgtcacttcacagttttgagactaacaaacacccttaggtctaccccaaaccaaaaaaagccagggagggacagtttanacaacttttaagntaagactagaatgcccccntaagtagataggcaccaccgttatacgcctttagcacaaggcttagaaaggcaaaacatttctaaagacataaaaccgccaaattgtcatttacactaagctgtggtattttttgttttgatttttaaaaattcctatctctaagtaatcaaatacagcaaaatttaaaactattctgccaaaccataccagttcaaaggtctgaaaccgttctttgctttggtgaatgtttggttaaaataaatcactgtttcactccatgagaagttttacatcagagcactcaattcacaggcaaaaaaaaaaagttnctttttaaatgaaagcgtttaaagaacactgctgaataaaaatatgtggtctttacaatgagtaacatatcatctatgaaaacaaaccatttaatcatataaaacaaagaccatatttttaaatcagatcctggacagtttaaacaaaaattttaatttgtggtttatttcataatcccaaactggatctttcatttcttcttctaaacagtccactatgaaacttttcttagaagcaattaacccaaaacaccaaaatcatttccctagcagttgctgctaataaactggaactgtatatcccaagtccaagaaagatgtgcaaatgaaatgcttcttgcttcacccttattatctggagcccctagagtcttattttccttttggacatgaattcaggaacagttcaatatacctatgatgaacgtgggaccgatccttgagcatcgctgcaacagcatcctcatgggtctcaaagtgcacatcagcttctccagtggccttcccactggagctgtattccatggtgattctaacaggcttgagtggagcaaaaaagtttataatgtcttgggcattggcttggaaaggtaatcctctcatgtggacaaaatgcagagaagacgtagttccaaaatcagcagcctctggaagcttttctggcacctccttaggcaattctatttccttctcactttcaaaagctgtcatgggttgaatatcctcatttacttcatgttcttcaaagaccatttctggctcagttatatacttagcagtaggaaaagatgcgattttctttcccttataagaaccgacatgtgttcgaacttcattccttctgcttggaaatatctcgatgtatcgattaccaatttcttccctgtgtttcaacagggcttggttggccatttctggttcttcaaattgcacataggcttcccctgtttttcgcctccctctatagtccatcacaaaagtaatgtcaactatattcagtcctgcaaagaagtctacaatgtctttctcattgcaactataaggaagtcctctcaaacgaaccacaccatcatttaccacaggcgaagatttgacctgcaagctcttcattaaggcatccacatcttcattgtttatctcatatacttccacataccgctggcccatgtacatacggtgcttctctaaggctttntgcacatcctgctctgactccatttcaattaaggcatcaccccttcgtttcccatctctgtttaggagaaaatgtattccattctcaccgttgcggattctgcagtctgaaaaaaagttaagcacatcttccatagtgcatgaccagggcagtccttgagctcgaatgagaaagacatcatccacttcctcttctaacttggacggggccaattcatactcagggggtggtggaaggtcttccaggtaagtagttttggactcctggctgtagctgcgcgtcgggacggcggccgcc

SEQ ID NO: 9 - DNA, 593 bp, Homo sapiens
  human ALDH1 cDNA fragment
tgaatatacagaggtcaaaacagtcacagtgaaaatctctcagaagaactcataaagaaaatacaagagtggagagaagctcttcaatagctaagcatctccttacagtcactaatatagtagattttaaagacaaaatttttcttttcttgatttttttaaacataagctaaatcatattagtattaatactacccatagaaaacttgacatgtagcttcttctgaaagaattatttgccttctgaaatgtgacccccaagtcctatcctaaataaaaaaagacaaattcggatgtatgatctctctagctttgtcatagttatgtgattttcctttgtagctacttttgcaggataataattttatagaaaaggaacagttgcatttagcttctttcccttagtgactcttgaagtacttaacatacacgttaactgcagagtaaattgctctgttcccagtagttataaagtccttggactgttttgaaaagtttcctaggatgtcatgtctgcttgtcaaaagaaataatccctgtaatatttagctgtaaactgaatataaagcttaataaaaacaaccttgcatgat

SEQ ID NO: 10 - protein, 17 aa, Homo sapiens
  human ALDH1 cDNA fragment
EYTEVKTVTVKISQKNS

SEQ ID NO: 11 - DNA, 418 bp, Homo sapiens
  human ferritin cDNA fragment
cggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccatgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgc

SEQ ID NO: 12 - protein, 139 aa, Homo sapiens
  human ferritin cDNA fragment
GPAGLSLASTVFGRNRSGDSLPASDRPPISSPLATSGTIFSAISCFWDLPAPFLWLAPSCQPTMSSQIRQNYSTDVEAAVNSLVNLYLQASYTYLSLGFYFDRDDVALEGVSHFFRELAEEKREGYERLLKMQNQRGGR

SEQ ID NO: 13 - DNA, 929 bp, Homo sapiens
  human ribosomal protein S2 cDNA
aaaacaccaaatggcggatgacgccggtgcagcgggggggcccgggggccctggtggccctgggatggggaaccgcggtggcttccgcggaggtttcggcagtggcatccggggccggggtcgcggccgtggacggggccggggccgaggccgcggagctcgcggaggcaaggccgaggataaggagtggatgcccgtcaccaagttgggccgcttggtcaaggacatgaagatcaagtccctggaggagatctatctcttctccctgcccattaaggaatcagagatcattgatttcttcctgggggcctctctcaaggatgaggttttgaagattatgccagtgcagaagcagacccgtgccggccagcgcaccaggttcaaggcatttgttgctatcggggactacaatggccacgtcggtctgggtgttaagtgctccaaggaggtggccaccgccatccgtggggccatcatcctggccaagctctccatcgtccccgtgcgcagaggctactgggggaacaagatcggcaagccccacactgtcccttgcaaggtgacaggccgctgcggctctgtgctggtacgcctcatccctgcacccaggggcactggcatcgtctccgcacctgtgcctaagaagctgctcatgatggctggtatcgatgactgctacacctcagcccggggctgcactgccaccctgggcaacttcgccaaggccacctttgatgccatttctaagacctacagctacctgacccccgacctctggaaggagactgtattcaccaagtctccctatcaggagttcactgaccacctcgtcaagacccacaccagagtctccgtgcagcggactcaggctccagctgtggctacaacatagggtttttatacaagaaaaataaagtgaattaagcgtg

SEQ ID NO: 14 - protein, 293 aa, Homo sapiens [sequence omitted]

SEQ ID NO: 15 - DNA, 525 bp, Homo sapiens
  human H2A histone antisense cDNA
ccgcttttttttttttttttaaaaaaaaaacaatcctttaataaaattagtccatctaaaactccccaatgcctaaggttctagtcgtggaagggttagctgcagaattccagttcagaagccaacggaggctcagtccagacagggattaaccgacttgtgctggtatctaggtgcttggattgccgagttgagtttgctggaagggaaatggggcggcgtcggggggcccgacaggcctggctgccagtcggcttccgcgaaaacgactcttgctgtccacatagccagccgtgaagccaaactgcaggcctctgccctcccctaaatgtcctcctaggaggcagtcactcgggaggaagatgtgcctgttaccaagtgcttcgtcccggccccagcgcagacctatgaatgaagatggagggagagctgatgtgaaaggcctgggtcccggccgcagaaggggcgccagaccgaaatcggcgatcgcgtctctcccaccaaagcctcccggccagcggccgc

SEQ ID NO: 16 - DNA, 1381 bp, Homo sapiens
catctggctgtaaggtttgcccttgaactaaaaatgttgtttggggcaagggccagaaatgtggagacatggtttttgttacgcattcttgtattatatgtgactaaatttacaaacaagatacatgtgtaattaaagacccttatggaactggaagacgtcttgtagtgctacattgggtgaaaccgttggtccatttttgtctgtttctatgaagataaaataattgggggccatctagaaatagaaaggcagtgggaagacagattctacggcactgctttcatttaattgggctttaggcactccattcgaatgcagaacctcacctctagttgagaccaagaattggcaaatttgcatgagctcctggaaagagttgctgactttgtatctaagacctgccagggaataccaagagttgtttctacagacttttttttttttttttgtatgggagaagatactgtggcaaccaggaaggaatggaaaaaaaattcttttctctacagcaaattaatgtgaggaagctcctccaatcctctggctatttaaggttcaaaatcaagtgcctagggaaaattccaatggatgattttctgggagctatcttgtctaccttgaggttcctgaacaatgaattcccattaatgagcagtcttcagtattaaaaccactgtcttgtcacctcattttgcattactgtcttccgtggatgtttcagttacaactgtaatgttatttatagaacaacattaatccattaaagctaacctatttttcaatatttatgataatctatgtacatatattgtctgtccatatgtatttgtaaataggttgtatataatgtcaggtttgggtcttgggttcaagtgtatatattcctgtaagtttcttaactgcattttgatgaattcacattatgtaactataagaattgtcccaaaagtacctgtacagaaaattgaatattgaaaaattgacaaattgtgtacaaacactaaaaaaaacttgtttaaattgtatttgcaataaacaacatcaaattttttcatgaaatcttggtacaaattcagatctcttatttaaaatttaaataaggaatacattttcaaaatgcagtaatcaaaatgtgatctagtgtaatgaaataaaatgtgatctagtgtaatggaagacctttgagaacctgggtgtattaactttgtgtatatagtgtaaatatccccactgtactgttagaggccaacaattctagtatggcttgttggcaaagagtgctacaccgtttcaatgaaacaatgtatgtttgttttaactgaactaaaataaatacatgcttaatcccgaaaaaaaaaaaaacaaaaaaaaaaaaaaa

SEQ ID NO: 17 - DNA, 2721 bp, Homo sapiens
  clone 11A12 - hypothetical protein DKFZp564K0822
gggctgctcttggagtgcacagaagccaaaaagcattgctggtatttcgaaggactctatccaacctattatatatgccgctcctacgaggactgctgtggctccaggtgctgtgtgcgggccctctccatacagaggctgtggtacttctggttccttctgatgatgggcgtgcttttctgctgcggagccggcttcttcatccggaggcgcatgtaccccccgccgctgatcgaggagccagccttcaatgtgtcctacaccaggcagcccccaaatcccggcccaggagcccagcagccggggccgccctattacaccgacccaggaggaccggggatgaaccctgtcgggaattccatggcaatggctttccaggtcccacccaactcaccccaggggagtgtggcctgcccgccccctccagcctactgcaacacgcctccgcccccgtacgaacaggtagtgaaggccaagtagtggggtgcccacgtgcaagaggagagacaggagagggcctttccctggcctttctgtcttcgttgatgttcacttccaggaacggtctcgtgggctgctaagggcagttcctctgatatcctcacagcaagcacagctctctttcaggctttccatggagtacaatatatgaactcacactttgtctcctctgttgcttctgtttctgacgcagtctgtgctctcacatggtagtgtggtgacagtccccgagggctgacgtccttacggtggcgtgaccagatctacaggagagagactgagaggaagaaggcagtgctggaggtgcaggtggcatgtagaggggccaggccgagcatcccaggcaagcatccttctgcccgggtattaataggaagccccatgccgggcggctcagccgatgaagcagcagccgactgagctgagcccagcaggtcatctgctccagcctgtcctctcgtcagccttcctcttccagaagctgttggagagacattcaggagagagcaagccccttgtcatgtttctgtctctgttcatatcctaaagatagacttctcctgcaccgccagggaagggtagcacgtgcagctctcaccgcaggatggggcctagaatcaggcttgccttggaggcctgacagtgatctgacatccactaagcaaatttatttaaattcatgggaaatcacttcctgccccaaactgagacattgcattttgtgagctcttggtctgatttggagaaaggactgttacccatttttttggtgtgtttatggaagtgcatgtagagcgtcctgccctttgaaatcagactgggtgtgtgtcttccctggacatcactgcctctccagggcattctcaggcccgggggtctccttccctcaggcagctccagtggtgggttctgaagggtgctttcaaaacggggcacatctggctgggaagtcacatggactcttccagggagagagaccagctgaggcgtctctctctgaggttgtgttgggtctaagcgggtgtgtgctgggctccaaggaggaggagcttgctgggaaaagacaggagaagtactgactcaactgcactgaccatgttgtcataattagaataaagaagaagtggtcggaaatgcacattcctggataggaatcacagctcaccccaggatctcacaggtagtctcctgagtagttgacggctagcggggagctagttccgccgcatagttatagtgttgatgtgtgaacgctgacctgtcctgtgtgctaagagctatgcagcttagctgaggcgcctagattactagatgtgctgtatcacggggaatgaggtgggggtgcttattttttaatgaactaatcagagcctcttgagaaattgttactcattgaactggagcatcaagacatctcatggaagtggatacggagtgatttggtgtccatgcttttcactctgaggacatttaatcggagaacctcctggggaattttgtgggagacacttgggaacaaaacagacaccctgggaatgcagttgcaagcacagatgctgccaccagtgtctctgaccaccctggtgtgactgctgactgccagcgtggtacctcccatgctgcaggcctccatctaaatgagacaacaaagcacaatgttcactgtttacaaccaagacaactgcgtgggtccaaacactcctcttcctccaggtcatttgttttgcatttttaatgtctttattttttgtaatgaaaaagcacactaagctgcccctggaatcgggtgcagctgaataggcacccaaaagtccgtgactaaatttcgtttgtctttttgatagcaaattatgttaagagacagtgatggctagggctcaacaattttgtattcccatgtttgtgtgagacagagtttgttttcccttgaacttggttagaattgtgctactgtgaacgctgatcctgcatatggaagtcccgcttcggtgacatttcctggccattcttgtttccattgtgtggatggtgggttgtgcccacttcctggagtgagacagctcctggtgtgtagaattcccggagcgtccgtggttcagagtaaacttgaagcagatctgtgcatgcttttcctctgcaacaattggctcgtttctcttttttgttctcttttgataggatcctgtttcctatgtgtgcaaaataaaaataaatttgggc

SEQ ID NO: 18 - protein, 168 aa, Homo sapiens
  clone 11A12 - hypothetical protein DKFZp564K0822
MPLLRGLLWLQVLCAGPLHTEAVVLLVPSDDGRAFLLRSRLLHPEAHVPPAADRGASLQCVLHQAAPKSRPRSPAAGAALLHRPRRTGDEPCREFHGNGFPGPTQLTPGECGLPAPSSLLQHASAPVRTGSEGQVVGCPRARGETGEGLSLAFLSSLMFTSRNGLVGC

SEQ ID NO: 19 - DNA, 4384 bp, Homo sapiens
  Clone 14H12  -   cDNA FLJ10838 fis, clone NT2RP4001274, weakly similar to Human transporter protein (g17) mRNA
agccgcgcgacgccgccgccttagaacgcctttccagtactgctagcagcagcccgaccacgcgttaccgcacgctcgcgcctttcccttgacacggcggacgccggaggattggggcggcaatttgtcttttccttttttattaaaattatttttcctgcctgttgttggatttggggaaattttttgtttgttttttatgatttgtatttgactgagagaaacccactgaagacgtctgcgtgagaatagagaccaccgaggccgactcgcgggccgctgcacccaccgccaaggacaaaaggagcccagcgctactagctgcacccgattcctcccagtgcttagcatgaagaaggccgaaatgggacgattcagtatttccccggatgaagacagcagcagctacagttccaacagcgacttcaactactcctaccccaccaagcaagctgctctgaaaagccattatgcagatgtagatcctgaaaaccagaactttttacttgaatcgaatttggggaagaagaagtatgaaacagaatttaattctcttgacatttgtgtcaatattttccctgtattctgttcatctccttttgaagactgccaatgaaggagggtctttattatatgaacaattgggatataaggcatttggattagttggaaagcttgcagcatctggatcaattacaatgcagaatattggagctatgtcaagctacctcttcatagtgaaatatgagttgcctttggtgatccaggcattaacgaacattgaagataaaactggattgtggtatctgaacgggaactatttggttctgttggtgtcattggtggtcattcttcctttgtcgctgtttagaaatttaggatatttgggatataccagtggcctttccttgttgtgtatggtgttctttctgattgtggtcatttgcaagaaatttcaggttccgtgtcctgtggaagctgctttgataattaacgaaacaataaacaccaccttaacacagccaacagctcttgtacctgctttgtcacgtaacgtgactgaaaatgactcttgcagacctcactattttattttcaactcacagactgtctatgctgtgccaattctgatcttttcatttgtctgtcatcctgctgttcttcccatctatgaagaactgaaagaccgcagccgtagaagaatgatgaatgtgtccaagatttcattttttgctatgtttctcatgtatctgcttgccgccctctttggatacctaacattttacgaacatgttgagtcagaattgcttcatacctactcttctatcttgggaactgatattcttcttctcattgtccgtctggctgtgttaatggctgtgaccctgacagtaccagtagttattttcccaatccggagttctgtaactcacttgttgtgtgcatcaaaagatttcagttggtggcgtcatagtctcattacagtgtctatcttggcatttaccaatttacttgtcatctttgtcccaactattagggatatctttggttttattggtgcatctgcagcttctatgttgatttttattcttccttctgccttctatatcaagttggtgaagaaagaacctatgaaatctgtacaaaagattggggctttgttcttcctgttaagtggtgtactggtgatgaccggaagcatggccttgattgttttggattgggtacacaatgcacctggaggtggccattaattggcaccactcaaactcaaactcagtccatctgatgccagtgttgagtaaactcaactactatgaaatttcacctaatgttttcagtttcacttccttttgaagtgcagattcctcgctggttcttctgagtgcagaataagtgaacttttttgttttgttttgtttttttaagaaacttatctgtatgttagaaatggatatgaacaacaaaaccacgagtctcgggttaagggaagtgacaattttattccattccagagaatggacaaactcttaacttttatcaagccacatgcttggctgtgtcattgtttaacttggatattttatgattttacttgaatgtgcctaatggaaccatttgatgtgagaaacaattctttttaatttacagcaaaatattgaataaccattgacaaaaacactattattttttgtaccaaaaatacttaaagacctcagaagcactcttttacttttaagaaattgcttttttgaactttattcagaagcagttatcaataaattccataaaataatgtcattggtatttaaaaatgaatattaatataatgaaatggtttgcctttttgtaggcataataagccaaatacttttttacccaaaataatttttagagaaaatgatgtaatgaaaaattgtaccatgaattaggagcatagttttttccatttaaacgtcaccattacttaaaagatgattgattgttgctataccaaatcagatgaactctgttcatcacttttcttctctgtccccaaacaatttggttcattcagactgaaatgtttgtgtcttcaacttattagaatggaagataatgcagatatttctgtgggaaataaaataactaattttgaggtaccaaatagtgcaattgggtaaaacagggtttattcagttgcatctgtctccagtgttgtattgacagctctgggtcttttttttgggccagcccttttttgacattgcttccagcagtggaaaatgggcatttgatggcaataggccaaaattattgtgtccagggagtacactttttcaaaatgctcacctactggaagtgtgaattacttgacaatgtatggcttagttgtgttcatgttttgtctacagtagaggtctaatccacaggttacacctatgtttgatatgatataagttctctttgcgtaggccactgggtttctcatgcagtaagctttataaaaactcatttgcactggactgtcatctcattcttgtacaacgtagaattacttgtttacatccaacaaatggttagctagggaaaacagtgcaaactgagtgttagtagtcattttggtccaactgcatgtcaacccttccatttcaatcccagttagaaatgaaaataattactttgaaacttggctttaagagcacatttatcgtacgtcacagtgtatggtgaatatattattaaataatgtggtacttcgctcatcaggcataatgtctaaaatctaatatacataattccattaagtggttgaaggaagcaaataatggaattgtcaattggtcatctggctgtaaggtttgcccttgaactaaaaatgttgtttggggcaagggccagaaatgtggagacatggtttttgttacgcattcttgtattatatgtgactaaatttacaaacaagatacatgtgtaattaaagacccttatggaactggaagacgtcttgtagtgctacattgggtgaaaccgttggtccatttttgtctgtttctatgaagataaaataattgggggccatctagaaatagaaaggcagtgggaagacagattctacggcactgctttcatttaattgggctttaggcactccattcgaatgcagaacctcacctctagttgagaccaagaattggcaaatttgcatgagctcctggaaagagttgctgactttgtatctaagacctgccagggaataccaagagttgtttctacagacttttttttttttttgtatgggagaagatactgtggcaaccaggaaggaatggaaaaaaaattcttttctctacagcaaattaatgtgaggaagctcctccaatcctctggctatttaaggttcaaaatcaagtgcctagggaaaattccaatggatgattttctgggagctatcttgtctaccttgaggttcctgaacaatgaattcccattaatgagcagtcttcagtattaaaaccactgtcttgtcacctcattttgcattactgtcttccgtggatgtttcagttacaactgtaatgttatttatagaacaacattaatccattaaagctaacctatttttcaatatttatgataatctatgtacatatattgtctgtccatatgtatttgtaaataggttgtatataatgtcaggtttgggtcttgggttcaagtgtatatattcctgtaagtttcttaactgcattttgatgaattcacattatgtaactataagaattgtcccaaaagtacctgtacagaaaattgaatattgaaaaattgacaaattgtgtacaaacactaaaaaaaacttgtttaaattgtatttgcaataaacaacatcaaattttttcatgac

SEQ ID NO: 20 - protein, 406 aa, Homo sapiens
  Clone 14H12  -   cDNA FLJ10838 fis, clone NT2RP4001274, weakly similar to Human transporter protein (g17) mRNA
MKQNLILLTFVSIFSLYSVHLLLKTANEGGSLLYEQLGYKAFGLVGKLAASGSITMQNIGAMSSYLFIVKYELPLVIQALTNIEDKTGLWYLNGNYLVLLVSLVVILPLSLFRNLGYLGYTSGLSLLCMVFFLIVVICKKFQVPCPVEAALIINETINTTLTQPTALVPALSRNVTENDSCRPHYFIFNSQTVYAVPILIFSFVCHPAVLPIYEELKDRSRRRMMNVSKISFFAMFLMYLLAALFGYLTFYEHVESELLHTYSSILGTDILLLIVRLAVLMAVTLTVPVVIFPIRSSVTHLLCASKDFSWWRHSLITVSILAFTNLLVIFVPTIRDIFGFIGASAASMLIFILPSAFYIKLVKKEPMKSVQKIGALFFLLSGVLVMTGSMALIVLDWVHNAPGGGH

SEQ ID NO: 21 - DNA, 588 bp, Homo sapiens
  clone 1F2 - hypothetical protein HSPC014 (HSPC014)
tctcttgnaagctccccaaaaaggccaacgttcgcagcgctgcggaagatgaatgccagaggacttggatctgagctaanggacagtattccagttactgaactttcancaagtggnccttttgaaagtcatgatcttcttnngaaangnttntctngtgnnaaaaatgaacttttgcctagtcatccccttgaattatcagaaaaaaatttccagctcaaccaanataaaatgaatttttccacactgagaaacattcagggtntatttgctccgctaaaattacagatggaattcaaggnagtgcagcangttcagcgtcttccatttctttcaagctcaaatctttcactggatgttttgaggggtaatgatgagactattggatttgaggatatnntcanngntncctctcnaagcgaagncatgggagagacacacttgntggnggaatntnanctcggctnactgtgatagngagctnggtcatggaaaccgagggntgaacntggnaatagnnatcttggccctgnantcgctgcaccacanccttnaaagtcctggcnnccctnaaaaaaaanaaaaaaa

SEQ ID NO: 22 - DNA, 815 bp, Homo sapiens
  clone 2H4 - hypothethical protein MGC2577 (MGC2577)
cccgaccccccaagcccactggtgaaacagctgagtgaagtatttganttctgaagactctaaatcaaatcttcccccagngnctgttttgcccccagaggcacctttatcttctgaattggacttgcctctgggtacccagttatctgttgaggaacagatgccaccttggaaccagactgagttcccctccaaacaggtgttttccaaggaggaagcaagacagcccacagaaacccctgtggccagccagagctccgacaagccctcaagggaccctgagactcccagatcttcaggttctatgcgcaatagatggaaaccaaacagcagcaaggtactagggagatcccccctcaccatcctgcaggatgacaactcccctggcaccctgacactacgacagggtaagcggccttcacccctaagtgaaaatgttagtgaactaaaggaaggagccattcttggaactggacgacttctgaaaactggaggacgagcatgggagcaaggccaggaccatgacaaggaaaatcagcactttcccttggtggagagctaggccctgcatggccccagcaatgcagtcacccagggcctggtgatatctgtgtcctctcaccccttctttcccagggatactgaggaatggcttgttttcttagacttctnctcagctaccaaactgtactgcacccattttggtggacgatgaaatggaaatagcccctaatatgtcaaagccaaaaataccctttttgaaaagccgccttggaagtcgngaatcacttcnttcacaactggatgatggttac

SEQ ID NO: 23 - DNA, 533 bp, Homo sapiens
  clone 3C2 - clone FLC0593
ggccaaccggccgctttttttttctgcttaaaaatttcaattctcgtggtaataccagagtagaaggagagggtgactttaccgaactgnnggccattggggaggcagatgcgggtntggaggtgtgggctgaaggnantgactgtttgattttaaaaagtgtgactgtcagttgtatctgttgcttttctcaatgattcagggatacaaatgggcttctctcattcattaaaagaaaacgcgacatctttctaagattctctgtgggaaaatgactgtcaataaaatgcnggtttctgggccattcgtcttactttcattttttgattacaaatttctcttgacgcacacaattatgtctgctaatcctcttcttcctagagagagaaactgtgctccttcagtgttgctgccataaaggggtnnggggaatccattgtaaaagtcccangttctaaattaactaaatgtgtacanaaatgaacgtgtaagtaatggttctacagggctttgcaacaaaactggcctttcgt

SEQ ID NO: 24 - DNA, 547 bp, Homo sapiens
  clone 3F11 - BAC clone CTD-2314H8
gaagaactngttctacaattcagctgtatgttgtaaaggagtcaagtcaatgaaggagttataaagagttgctgaaaaatggagnaacagtgtatgggacttcacatcacntctccttattgattgttannttgatccctcttgttcttttgttgtaaacattttctataattaggaaatgccatttaagagtgagagaggtatatatctatgagccattgtgtttggtgttttacaagaactttaccatactggtgtgtagtccattctgtacagtttaaaagtgattcacgatttgcaggctttttatcagatcacaaaaaaatcagtctttaagcatttgcttggtaaggtttcttaagattaggtttataatacaaccatctgtaatgtatctctcgtttgagcttgtgggccatacaattcattaactagatgaatacattgtggacagcatcctcactacccctctctactcactcacaaagaaccatgatacactggaatgtttttctctggaatcctctttctactcttgnattaaaat

SEQ ID NO: 25 - DNA, 537 bp, Homo sapiens
  clone 4D10 - full length insert cDNA clone ZE03C06
gcttggccnaaaaggccaactaaaagcctacgtgttggtaaatccaacagcaagggagatttttgaatcataataactcataaggtgctatctgggnagtgatgccctcagaggnnttgctgttagctggcagctgacgctgctaggatagttagtttggaaatggtacttcataataaactacacaaggaaagtcagccaccgtgtcttatgaggaattggacctaataaattttagtgtgccttccaaacctgagaatatatgcttttggaagttaaaatttaaatggcttttgccacatacatagatcttcatgatgtgtgagtgtaattccatgtggatatcagttaccaaacattacaaaaaaattttatggcccaaaatgaccaacgaaattgttacaatagaatttatccaattttgatctttttatattcttctaccacacctggaaacagaccaatagacattttggggttttataatgggaatttgtataaagcattactctttttcaataaattgttttttaattt

SEQ ID NO: 26 - DNA, 582 bp, Homo sapiens
  clone 4E9 - hypothetical protein dJ465N24.2.1 (DJ465N24.2.1)
tggccaaaaaggccaaccctgtctagctgtcggaaaaggtaacagaagatggaactcgaaatcccaatgangnacctacccagcaaagangggtagcttttagctctaataggttctgtagcaaagccaatacaaaaatcagctaaagctgccacagaagaggcatcttcaagatcaccaaaaatagatcagaaaaaaagtccatatggactgtggatacctatctaaaagaagaaaactgatggctaagtttgcatgaaaactgcactttattgcaagttagtgtttctagcattatcccatccctttgagccattcaggggtacttgtgcatttaaaaaccaacacaaaaagatgtaaatacttaacactcaaatattaacattttaggtttctcttgcagatatgagagatagcacagatggaccaaaggttatgcacaggtgggagtcttttgtatatagttgtaaatattgtcttggttatgtaaaaatgaaattttttagacacagtaattgaactgtattcctgttttgtatatttaataaatttcttgttttcattcttaaaaaaaaaaaaaaa

SEQ ID NO: 27 - DNA, 388 bp, Homo sapiens
  clone 4F7 - chromosome 1 clone RP11-109I2
tttttttnccctggatcagctcggcctnnaaggcctcggaaaacaagatgcttcctctggaatgtgagtccaaagagttaccagcgctgccctctagtgatctcagctcaggntatgcactaaccgtgtggntacagggctgagtagtgctgcagtgtgaagtgaatggaaggcctcgaggtgtttgtggctggccaccctgatcagcctgcaggtagtcccgatgaagccagggcacagggggattcgttccagcttgttcactttattctgccttgccaggttactgaaagtccctcgtttgctctcaccagccttcctggaaatgtggactcttgaaagaaaagctcccgtgctcttgaagtatacctgcttgccangggagtcc

SEQ ID NO: 28 - DNA, 605 bp, Homo sapiens
  clone 4S-10d4 -DNA sequence from clone RP1-64K7 on chromosome 20q11.21-11.23 Contains the EIF2S2 gene for eukaryotic translation initiation factor 2 subunit 2 (beta, 38kD), and a putative novel gene.
tgcctggtcatgctggcaccgggtcatatgctggacagggagaacgagagtcccatcctggaactccagaaaagcccctggatgctccagcccctgggaaagcacacagccaggcccttgggtgggaggttggcttctaacagtgcatacacatgcccttcctctgagtcggggcagcaaaaacatccattccgctgcgcaacagttgtcatttttctaacatctgaaaactccagaaggagatggtgataaatgtggtaccggattctgcctaaaggatcagtctttagatgttttcagattgaaagcctcatttgtgatcctcacagccatcttgaaagaatagagcagccagtgggtatactggattgtgagctaagaggcctgggactttccccctgttgctgccagccaggttgatgaccctgggcaagtctttttccttaccaggtctcagtttcctcagctgtaaaatgagaggttgatctggatcagggatagtaaatgggcctttgttcagttactgactgttgtataacaaaccacccccaaatttagtagccttaataaacatttattagctcctgaaaaaaaaaaaaaaaaaa

SEQ ID NO: 29 - DNA, 661 bp, Homo sapiens
  clone 4S-10G6  -  similar to C50F4.16.p (LOC256281)
tggggtgagtggccaagactggcctctgtctagaaccctggagtctcactggagatccaggttgggggccacctggctgaggaaccatgagacaccaaagatgacgccgagggtcttggggatgtccgggtcgtctgcaaaggcctgggcgccttccaggggcaggtgcaccacctcaatgagctcaccctcctccaccaggcccccacctggaccgctacgctgggcatctgtcacctctgtgtagaacatggtctgtctggagccagtcagtcccactccagaccagtatgtggcgacccggcgcagatcagagggggccaagtggtagccacactcctcccaagcctccttgcaagccacttcctccagcgagagcccaggctggtccacgaggccggcacacagctcaactgtcacccccgctgagccgggcagggctggctgtagctcccgaggcccgtcctggtctacagctgctagggaccctgggaagcggcgctccacctcacccgcatacacagctggccggaactgcttcaccaacaccaggctcctccgagaagagttgaataagagaacggtcacgctgtcatgcgtcttcatgaagtcccaggacttctgggcaccattctggcggtaatgcagcgtgagcggccgc

SEQ ID NO: 30 - DNA, 667 bp, Homo sapiens
  clone 4S-11B8 - DNA sequence from clone RP11-735A5 on chromosome 1
tttttttttttttttttaaagaaaacctactgtatgccatttgcaagagatataccaaaagcaaactgatatgaaaaggctgaaataaagggatagttggtttctggaaaaactaacaacaacaaaaaaaatcttccatgaatttttgttgttgttattttaattttttttgagacacggtcttgctgtcacccaggctggagtacaggggcacaatcatggctcacggcagcatcgacctcctaggctcaaacgatcctcccactttagcttcccaaatagctggaactacaggtgtgcaccgccacgcctggctacttaaattttttttttttttaatttgtacagacatctcaccatgttgcccaagctggtctccagctcctgggctcaagtgattcacccacctggcactctcaaagtgctgggattataggcatgagccactgtactcggcattccactgatacttgataatgaaaaaaaagagtcttaatacccaatattagaaatacaaagagaaacagaagaacaaatcagcaaaggtttaaaagaacaaaaattttatatatatatatatataaacaaattgatctcaataaataacaaaataaacaaaatggacaaactcttaaaaacatataccttaccaaaactacctcaaaaa

SEQ ID NO: 31 - DNA, 578 bp, Homo sapiens
  clone 4S-11H3 - cDNA FLJ14279 fis
ttgtcaaaaatccattcctgtcccctccaccctgtttttattcctctatccactggaaaccattaaaaaaaaaagtttttgatttatagtcaattcttgtatttgaggtagttgtgttctataaagtgtggcaaatactgaattagcaaataccaaattattactactagggcaaatacacgattaagtttctgtgagcctctggtaatatttttgtcaaccaatcgatacatttactttgttttatgagtgtttctgttaaagataccttatttagtatatattgttgattcattaacgtttaactcactgccaatagtattaaaactcatatgaatgaaatgtgtctaacacgtattttccctataagtcatatcatggccttcttgagcttaagaacactagagagcacttcagcactacacttgagggccattttatttacttatttttacagatggatcttgctatattgcccaaactggactgtaatggctattcacaggtgccaaagtagcatactgcagactcaaactcctggcctcaagccatccacctcatcctcccaagtagttggg

SEQ ID NO: 32 - DNA, 352 bp, Homo sapiens
  clone 4S-12A9 - chromosome 18, clone RP11-13N13
acatcacttgaggtcagtagacagggtttcaccatgttacccaggctggtctcgaactcctgacctcaagtgattcgcccatctcggcctgccaaagtgctgaggtcacaggcatgaggtcaggagaacgagaccatcctaacacagcgaaaccccgtctctactaaaaagacaaaaaattagctgggtgtggtggcaggcacctgtagtcccagctactcgggaggctgaggcaggagaatggcctgaacccgggaggcagagcttgcagtgagccaagatcgcgccactgtactccagcctgggcaacagagcaagactgtctccagaaaaaaaaaaaaaaaaaaaaaaa

SEQ ID NO: 33 - DNA, 469 bp, Homo sapiens
  clone 4S-12E6- hypothetical protein MGC955
gggagagtggtcagcagtatttttccatcatttctccaaaggaatgggggacaagttgtccacatgacttccttggtgcctacaaactacagcatgacttgtcctggactccgtatgaggacattgagaagcaagatgctaaaatcagcatgatggacatgttgctaagccagtcagtggccctgcctccgtgcactgaacccaacttccagggactgactcactgagagtgggctttgacaaacagctctcacaggacctggctgtcaacctccttgttgcccccactgttgccttgagaattgaagacatgtaggtgactcacaaacttcttggaaagagaccctgtgtgaatgtaaatgctgtcattatgacttttaattgggatgggaataatcattgagacagagtcactgtctttcgggatcctctttggaccacagatacccaagtcagtcagtttcagagt

SEQ ID NO: 34 - DNA, 795 bp, Homo sapiens
  clone 4S- 14D4 - clone IMAGE; 4778940 mRNA
aaaagcagatttaaaaagcagctcacagttgggcgcggtggctcacacctgtaatcccagcactttgggaggccgaggcgggtggatcaccagaggtcagaagttcgagaccagcctggccaacgttgtgaaaccctgtctctactaaaaatacaaaaattggccacgtgtggtggcgggcacctgtaatcctagctacttgggaagctgagacaggagaattgcttgaacccgggaggtggagtgcagtggcatgattaaggttcactgcagcctcaatctcccactctccagcgatcatcccacctcagcctcttggatagctgggaccacaggcacgagctaccatgcctggctaatttattttttgtagagacggggtttcgccatgttgcccaggctggtctggaatttctgacctctggcagtccacctgtttccgcctcccaaatgctgagattagaggcatgagtcactgcacccagcccgcagcctcttttataagggcattaatccccttcatgagggatattctctcatgacttaatcacctgccaaagaccccacctcttaatactacattaagtgatgggttcaatgtattnaattggggggggagggggcacattcagaccatagcatcttagtcattcttggttttatttaagattatttagactganggcattgaaaaatagcatacttggatgggacttcagcattcnatnagtggccttaataaaacctggtaattaaaaaagcncttaattttgcaaaaaaaaaaaaaaaaaaaaa

SEQ ID NO: 35 - DNA, 324 bp, Homo sapiens
  clone 4S-2A6 - PAC clone RP3-414A15 from 14q24.3
tggcgtgaaccccagaggtggagcttgtgagaggtgacagcgtgctggcagccctcacagcacttcctctgcctgggctcccactttggcggcacttgagaagcccttcagcccaccgctgcactgtgggagcccctttctgggctggccaaggccggagccgtctccctcagcttgcagggaggtgtggagggagaggcgcaagtgggaactgaggctgcgttccgcacttgcgggccagctggagttccaggtgggcatgggcttggtgggccccacactcggagccgccagctggccctgccagccccgggcaatgagggg

SEQ ID NO: 36 - DNA, 226 bp, Homo sapiens
  clone 4S-2D3 - DNA sequence from clone RP11-357H24 on chromosome 10
gtctgctgcctactcgtatgtaatatgtgtacataaaagcggcagctggtttttcgtttaagagtaatctaatatacagaatttgggcccttaagggtttatacctcttcatttaaaatgctttctggacaatctgctaccaaacacattttgttataggtgacattaaaactacatacaaatctacctgcacgacaacacataaaaaaaaaaaaaaaaaaaaaaa

SEQ ID NO: 37 - DNA, 377 bp, Homo sapiens
  clone 4S-2E2 - cDNA FLJ14385 fis, clone HEMBA1002212, weakly similar to TYROSINE-PROTEIN KINASE 2
gttggcctttgtttaaaacactgaaccttttgctgatgtgtttatcaaatgataactggaagctgaggagaatatgcctnaaaaagagtagctccttggatacttcagactctggttacagattgtcttgatctcttggatctcctcagatctttggtttttgctttaatttattaaatgtattttccatactgagtttaaaatttattaatttgtaccttaagcatttcccagctgtgtaaaaacaataaaactcaaataggatgataaagaataaaggacactttgggtaccagaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - DNA, 758 bp, Homo sapiens
  clone 4S-2F8 - chromosome 17, clone hRPC.1110_E_20
caaaaaaaaaaaaaaaaaaaaaaaaacatcagatcttgtgagacttattcactatcaggagaccagcacacgggaaagaccctcatgattcaattacctcccaccaggtccctcccacaacacataggaattatgggagctgcaattcaagatgacaattgggtggggacacagcaaaaccacatcacatgctgagctgtagcaggtgaataaaccactgagactacgaacctctgtcctctgaagaagatgccattttctcaaattctaggagtgtgggccactgttctgaggttgctgcagaggagcactgctggagaaaaggaagggggaaatccacatatccactaagacatcgtcagaatactgcacatggaattataggatattaacaatttaagagactgtagacacaggatagtcaaaatctcccattttacagacaaatcaacagaggcccagagcagtgaaggcatttacccagacactcactggatcggtgcagctggatctagatccaggtctcttgactcatttaacagctgttaaaccaaaaatgggtgtgatttagtcccattgtcatctgatacattggcaatgccctgcatatttttttgtctctatgtttaatactcctttgtaaagggtagcttttgatatttcctgaactgagcatctgttaaaattggattcatcttccctttaaggcaacaattggtgtttctgatctttaatgccaaaaaaaaaaaaaaagaaaa

SEQ ID NO: 39 - DNA, 840 bp, Homo sapiens
  clone 4S-3C2 - clone FLC0593
tgactttaccgaactgacagccattggggaggcagatgcgggtgtggaggtgtgggctgaaggtagtgactgtttgattttaaaaagtgtgactgtcagttgtatctgttgcttttctcaatgattcagggatacaaatgggcttctctcattcattaaaagaaaacgcgacatctttctaagattctctgtgggaaaatgactgtcaataaaatgcgggtttctgggccattcgtcttactttcattttttgattacaaatttctcttgacgcacacaattatgtctgctaatcctcttcttcctagagagagaaactgtgctccttcagtgttgctgccataaaggggtttggggaatcgattgtaaaagtcccaggttctaaattaactaaatgtgtacagaaatgaacgtgtaagtaatgtttctacaggtctttgcaacaaactgtcactttcgtctccagcagagggagctgtaggaatagtgcttccagatgtggtctcccgtgtggggcccagcaatgggggcccctgatgccaagagctctggaggttcttgaaagaggggacacgaaggaggagtgactgggaagcctcccatgccaaggaggtgggaggtgccctggaaatagctgcctcatgccacttaggccatgactggatttaatgtcagtggtgtgccacagtgcagtggctagacaactgaaaggggctaccaaggctgggaaaaaaatgcaattgttgctgtgagtgactttgaaagactctggtgccttgtggtgcccttctgaaattcaaacagtaatgcaaaagtgtctgcattagaatttacggtgtc

SEQ ID NO: 40 - DNA, 480 bp, Homo sapiens
  clone 4S-3F11 - BAC clone CTD-2314H8
agagttgctgaaaaatggagtaacagtgtatgggacttcacatcagttctccttattgattgttagtttgatccctcttgttcttttgttgtaaacattttctataattaggaaatgccatttaagagtgagagaggtatatatctatgagccattgtgtttggtgttttacaagaactttaccatactggtgtgtagtccattctgtacagtttaaaagtgattcacgatttgcaggctttttatcagatcacaaaaaaatcagtctttaagcatttgcttggtaaggtttcttaagattaggtttataatacaaccatctgtaatgtatctctcgtttgagcttgtgggccatacaattcattaactagatgaatacattgtggacagcatcctcactacccctctctactcactcacaaagaaccatgatacactggaatgtttttctctggaatcctctttctactcttgtattaa

SEQ ID NO: 41 - DNA, 506 bp, Homo sapiens
  clone 4S-4C11 - similar to putative, clone MGC33177 IMAGE4823662
atcatttgataatttaccttagagcatttaaaaaaatataatcaaactaattgccagccaagtcagtcatcctcctgggagtatatagagtcccaaggttagcgctcctgtattagactatttcaattttaggaaaatcatgaccatgtggggaaacaatgactttaaaatgctgaaattaaaatttatgctttaactggaatattttttgcttaactactcaattagaatattgtacacctgatcaatgtgtgttcagcacagatggccatgaattgtcatttatagtccaattttttatcttaatcataaaatgtttaggaatctatgaaatttaactttaggaacaaaacgtttagcagggttgattgatattatttttacattgttctggcaatccacagaaagagaagagccttaatttttaaaacccattttagtcattttatgacaattaaagttgtttaataaacatcttttttcaaagaagcaaaaaaaaaaaaaaa

SEQ ID NO: 42 - DNA, 558 bp, Homo sapiens
  clone 4S-4E9 - hypothetical protein dJ465N24.2.1 (DJ465N24.2.1)
ctgtcggaaaaggtaacagaagatggaactcgaaatcccaatggaaaacctacccagcaaagaagcatagcttttagctctaataattctgtagcaaagccaatacaaaaatcagctaaagctgccacagaagaggcatcttcaagatcaccaaaaatagatcagaaaaaaagtccatatggactgtggatacctatctaaaagaagaaaactgatggctaagtttgcatgaaaactgcactttattgcaagttagtgtttctagcattatcccatccctttgagccattcaggggtacttgtgcatttaaaaaccaacacaaaaagatgtaaatacttaacactcaaatattaacattttaggtttctcttgcagatatgagagatagcacagatggaccaaaggttatgcacaggtgggagtcttttgtatatagttgtaaatattgtcttggttatgtaaaaatgaaattttttagacacagtaattgaactgtattcctgttttgtatatttaataaatttcttgttttcattcttaaaaaaaaaaaaaaaaaa

SEQ ID NO: 43 - DNA, 352 bp, Homo sapiens
  clone 4S-4F7 - chromosome 1 clone RP11-109I2
gggaaaacaagatgcttcctctggaatgtgagtccaaagagttaccagcgctgccctctagtgatctcagctcagcatatgcactaaccgtgtgtttacagggctgagtagtgctgcagtgtgaagtgaatggaaggcctcgaggtgtttgtggctggccaccctgatcagcctgcaggtagtcccgatgaagccagggcacagggggattcgttccagcttgttcactttattctgccttgccaggttactgaaagtccctcgtttgctctcaccagccttcctggaaatgtggactcttgaaagaaaagctcccgtgctcttgaagtatacctgcttgccaggggagtcc

SEQ ID NO: 44 - DNA, 524 bp, Homo sapiens
  clone 4S-5C4 - similar to KIAA0674 protein
cccttccctaagcatgattttgcacagccaaccctgggtctaggcgaaccacagggtgaggtcaaggtgagcattctgggaacaatatttgggctcagagggtgggttggccaccttctgagccccacccccgccagacctggtgaagaggatcataaccctgtcttcaagaacactgggatttcagcagcaagttggaagaaggactggtaggttcccctccaagccagtcacctgtaagagtcctgtcctctgccagactttttaatctcttcattaactctcagactgacctgggagccctcctctacctgaatccagtgctcaactgtgccccggcaacaagacctgggctgaggtctccctggtagaactaagggagattacaccatctaaatcccagtgcagtcaacagcctggcctatagtcctgggacatgtatcttcttctttgccttaaatctgatacaagaggtcaatgactttgaaaataaaactaaaataaatgtcaaaaaaaaaaaaaaa

SEQ ID NO: 45 - DNA, 891 bp, Homo sapiens
  clone 4S-6F6 - similar to RIKEN cDNA 1110012M11 gene
atggattccaaggcctctgtgtccaagaagaaacgcatgtgtgtgaagctgttgcccctgggagccacggacacggctgtgtttgatgtccggctgagtgggaagaccaagacagtgcctggataccttcgaataggggacatgggcggctttgccatctggtgcaagaaggccaaggccccgaggccagtgcccaagccccgaggtctcagccgggacatgcagggcctctctctggatgcagccagccagccaagtaagggcggcctcctggagcggacagcgtcaaggctgggctctcgggcatccactctgcggaggaatgactccatctacgaggcctccagcctctatggcatctcagccatggatggggttcccttcacactccacccacgatttgagggcaagagctgcagccccctggccttctctgcttttggggacctgaccatcaagtctctggcggacattgaggaggagtataactacggcttcgtggtggagaagaccgcggctgcccgcctgccccccagcgtctcatagtccctcacccttccgcggaaagagcccccttactccacctccccgccagcctggggccaccccccctcactgcatcctgggaaccttcgccctgcaaggcgtttgctatcttcagccactgggcggagctgcagccctggaggagggggcgggtcgaggctgcgtggtgatggggtctccgcccccacgccctgccgggcagggctggagctggacagaagccagtgcctttaagtcatttgtgtcaaaaccctctggggtccggaggctgtgcgggtgtcctcctggcaataaacactacccggttctcgccaaaaaaaaaaaaaaaaaaaaaaaaaaaaaaaa

SEQ ID NO: 46 - DNA, 902 bp, Homo sapiens
  clone 4S-6H8 - cDNA FLJ31039 fis, clone HSYRA2000221
agagattctctaaatatggaattagattaggattctctgctccacttaacacacatttttaaattagtactgatgattgagggatggacaatagcacaccaaaaaaaaagagtttagtatgaaaaatttaaacctgttggttaagtcattcccattaatgtcattttgctgagggtgacttggtccttttgaattgctttggtgtacgggtatgttctgatttttcatgcaagctcctctgccattccaccgctctgaggagtaattgtagcacttcacatgtgctgtggttgtgatcacatggtgacatacatagcatgtgtgttcccagctgttgtgtgtttatgtgacatttgatgccaatacatatgtcttcaaggtatgcttgttccctcccagctcgtggaatatcaaaaaaattcattgctggaaaaattatttcatagacaaaaatgttaatgttctcttggggacttagagttgaaaatatttgtatagatttggttctcaagtccacagaatcgtatctgctgtggtctccctttggtgctcatctgggagccatgtgtatggaagattctgtcacaggcggctgggatgtgggcagatgctgttagcctccctctccacgtggtggtccatgcctgacgtgtcccctagttcaaggaagcgccatctttagcatgaaaacaattgcgttcccctaggaaatgaagaaaaaatgagctgaaatttccttatacatttgaatttgttcattttttaaagagacatttttgttgtctgctttgtggtacttataaaatttgttttccattgaaattgccatttataaatttgcagatatgtactaatttagattttttttaagtgttcaataaaataaggatatatttactgtg

SEQ ID NO: 47 - DNA, 566 bp, Homo sapiens
  clone 4S-7F6 - cDNA FLJ13305 fis
aagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaactgaagaaaatcttggtatggtgatgatttttactctagtgacagctgtgcaagaaaaattaaatgaaatagtagatcagataaaaactagaagagaagaagaaaagaaacaaaaagaaaaagaagcagaagaagctgaaaagcaattattccatggtactccagttacaattgagaatttcttaaattggaaagccaagtttgatgcagaactcttgg

SEQ ID NO: 48 - DNA, 482 bp, Homo sapiens
  clone 4S-7G6 - KIAA0776 protein (KIAA0776)
ttgatatctactgaaacataaatgataaggttcttaaaggttgaattaaaagtaatccatgtttgtgtcaaatgatcatagaaaataaatagaagagacagtgaagcaagtaaaaagaaaagcattgttttaatttgtttgcattaatttttttcatttgtcaaaatgcttcttttgttgccacagtaaagaacagtttttattgttttgtaagtaaaattacgtagctgattttgtatgtaaagattaatttccataataaaaattattgtatgtttactgtgatcttaatgggcagggttaagaaagttatttaaaataaagttacctattctactaaattttatagtactttgaagcttctattaattaacacaaagattaattggtgcatatattttatatatatacattttgaattctcattttgaacattattaaaggattttatttttcttacacaaaaaaaaaaaaaaaaaaaa

SEQ ID NO: 49 - DNA, 274 bp, Homo sapiens
  clone 4S-9F4 - similar to hypothetical protein FLJ10751
agatgagggttaggtgtgcccagccctccagacccggcctttctggttaacccctgcatgccaagctgcctgctgccccaggtcctcacctcaggcctttgaaggggcagcttctggaagttgttttctcctctgcttggagagtttgcccttgtctgtcttggaaagtgtgggcagccacagatgcccccaaatcagagctcacagtgagtgagcccctaagcttcagtctgcaataaagaatgcattggtttcatcaaaaaaaaaaaaaaaa

SEQ ID NO: 50 - DNA, 1021 bp, Homo sapiens
  clone 4S-9G10 - hypothetical protein BC013073 (LOC92703)
tgagagaactgtctccagaaaaaagaaaagcaagagacacaaagaagaactggacggggctggaggagaagagtatcccatggatatttggctattgctggcctcctatatccgtcctgaggacattgtgaatttttccctgatttgtaagaatgcctggactgtcacttgcactgctgccttttggaccaggttgtaccgaaggcactacacgctggatgcttccctgcctttgcgtctgcgaccagagtcaatggagaagctgcgctgtctccgggcttgtgtgatccgatctctgtaccatatgtatgagccatttgctgctcgaatctccaagaatccagccattccagaaagcacccccagcacattaaagaattccaaatgcttacttttctggtgcagaaagattgttgggaacagacaggaaccaatgtgggaattcaacttcaagttcaaaaaacagtcccctaggttaaagagcaagtgtacaggaggattgcagcctcccgttcagtacgaagatgttcataccaatccagaccaggactgctgcctactgcaggtcaccaccctcaatttcatctttattccgattgtcatgggaatgatatttactctgtttactatcaatgtgagcacggacatgcggcatcatcgagtgagactggtgttccaagattcccctgtccatggtggtcggaaactgcgcagtgaacagggtgtgcaagtcatcctggacccagtgcacagcgttcggctctttgactggtggcatcctcagtacccattctccctgagagcgtagttactgcttcccatcccttgggggcagcctcgagtgtagtccattagtaatcagattccagtttggacagggtggctggattgtatatctcgttagtaatgtacatgctcttcaggttctagggctcctgttaggggagggagaaatgttgaatcaagagggaaaacaactactatgatttataaacatattttaatgtaaaaatttgcattt

SEQ ID NO: 51 - DNA, 1343 bp, Homo sapiens
  clone 5S-21/57 - hypothetical protein FLJ10081
agatgattgacagtgactctactatgcagggctgttggtaccaacctgagccctataggtggcagtccctggagaagtggtcacagaagatggagctctgatcccctgcttacctcttcacaacacttgtgtgcaaagatagttttagatttggtttagaagctatcctccagaacaggctcccatacttagaatgtttctagttaaggtaataaattaggcaacccaagtgtgactccactcaagtgtccttttctgtaggcaggaagggcccacaacatggcttaaaatgtagtccatggttctggcccacagtacagtgtgtatctataccaggtcacctgtgttcaatctgggagccttcctggccagtctgagtggcagccagaagggagctcatagtgtctaggagtctcargcaaggtaggtcagggtactgtgggcaggggggatgtgtgtgataggagagggtaccctaaaccccataccttccctccctgacctgaaaaagctgatcttcaacagggattcacacagaattaggctgtgtttttgcattagctggtaggtgactttctcaaaattcttaaattcagaaagtatttantaaacttgaggaaggtatgaaatctggaggaggcatccaggacccaggggtttgatagctttacaggtaggatcatacccacccaaaagagcagtggacaataagactatttgagctatatgaagcttttaggaatcatttaggacagacagagcccttaamcaacccattcatgacttaagttgttggcttcartgtwtkcctggggacaaagaaaaactaacaagcccgacctgcctttatgataaattctagtgtgctwacaagggatgacttcctgaggtgtgatctgtccaccttgaagaactccacaactgaagaaggggagctgtgagaacgtggattgttctacaacttgcacagggtaacagaggaagtggctgaggcctagagtcacgttttccagttcccttcgcaaactatatttcttggaacgcgaaaggaagctttacctatttcatagaagacctggaatccataacctcagaaggcaatattattgatagaaaatgtggaaggatcaggaagttcttagattcttggatgacanatgcatgttgatgccctatggagatgtccttgtgttttgaggtcactgaggtaggaagacctgtctactcttggtttcaccactagaacagtcttgggctggatgggttatagagctgagcggctgtgatggttctgtttttacattaacaaaaacaattaaaaacaccaaaaacaaaaaaaaaaa

SEQ ID NO: 52 - DNA, 2073 bp, Homo sapiens
  clone 8C10 - hypothetical protein FLJ23018 ( FLJ23018)
aagaaaattcgagggaaagaagtttacatgactatggcttacggcaagggagaccccctcctcccacccaggctgcagcacagtatgcattatgggcacgatcctccaatgcactactcacagacagctggcaatgttatgtctaatgaacattttcatcctcagcatccatctccgagacaaggtcggggatatgggatgcccaggaattcatctcggtttataaacaggcacaacatgccgggccctaaagttgatttttacccaggcccaggtaaaaggtgctgccagagctatgataacttctcttatagatctcgttcatttagacgtagtcaccgccagatgagttgtgtgaataaggagtcccagtatggatttaccccagggaatggacagatgcccaggggcttggaagaaactattactttttatgaagttgaagaaggggatgagactgcttatgcaacttttcctaatcatgaaggtccctctacaatggttcctgctacttcaggatactgtgttggaaggcggggacatagctcaggcaaacagactttgaatttagaggagggcaatggccagagtgaaaatgggcgatatcatgaagaatatctttatcgtgcagagccagactatgaaacttcaggtgtttatagcacaactgcatctacagcaaacttgtctcttcaggacagaaagtcatgttctatgtctcctcaggacacagttacctcatacaactacccccagaagatgatgggaaatattgcagcagttgcagcttcctgtgccaataatgttccagctccagtcttatctaacggtgcagcggctaatcaagctattagtaccacttcagtttcctcacagaatgctatacagcctctctttgtatctccacctacacacggcaggccagatacaaaagttttgcagtactatttcaatctaggattgcagtgctattaccacagctactggcactccatggtctatgtgccacagatgcagcagcagcttcatgtagagaattatccagtctatactgagccacctctggtagatcaaaccgttcctcaatgctacagtgaggtgaggagagaagatggcatacaggcggaagcatcagcaaatgatacttttccgaatgctgattcttcatctgtccctcatggagcagtctattatccagtaatgtcagatccctatgggcagccacctttgccaggttttgactcctgccttccggttgtgccagattattcctgtgttcccccctggcatccagttggtacagcatatggtggttcttctcaaattcatggtgctataaatcctgggccaattggctgtattgctccatctcccccagcttctcattatgtacctcagggtatgtaagatccagcagtatgaagtattcttgcactgccattttcttgctgtttttgtttttaaaaagtattttatgttagtggttaaatgatttaggtgattagtgtttactattgtatttgtctttaaaattattttatcttttgatttaaaatagtactttaaaattaaggggtattattttgggctgtgactaaggaaattgagatggatgtacaactagccccatattgagcatacttcattgtattcagctgttttcctgtcagccatttgtcagctttatattagctgatggtaccaattgataaaatgaatataaagtatttcattggttcaaaaatcacacatcatattaaaccatgcagaattggagtaacttccacttttttctagaaagtaaaaccaagagcctttgcttctggataactcacttaatattaaattaaagagctcttcacgtttcttgagaattatctgaagccagttgcattctgtgatatcagttttgaaggcacatggttctctgctttagatttatcccatatgctattgtttaatactggatgtatgtaagtgttttactgcactgtattgaattggtgtcttttgcacagttagcagtaaataaaaattagcatttaaaattgcaaaaaaaaaaaaaaaaaaaaa

SEQ ID NO: 53 - DNA, 804 bp, Homo sapiens
  clone 5D-15/114 - cDNA DKFZp434O159
tatgattgaaatgcattcattcatcacgcataggcacaatcacaacttgatgatgcttgggaaagaatcaacagttaaaacttcatgaagttctaatgtctgtgttccaaaacacatcacattattaggttgtagggacatacgtaggtgtgctccctggggtggggagttttctagttactagaccatctcccatttttagcacttggcagcctcatgatccttttataaataggagattaacaggagagcagcaatacgattttgccaatggaataacagatttgccggcattcactgaaagagggcasatattgggtccttgtgacttcaactgactcttccgaattgtatgaatttatcaatgtattagataaacccagtttcagaataataaagaaaaaatattagaccaaataatgtggctaatagtgggtatgatttctagcccgtgggtttaaaactgtatcctaaagagtcattttaaaataatatwaatatttaaaaatgtaactgctatctttatgttctgaaataagttaaaacattttaaaatatgaatactgtagtttaaaagaaagaaatggtgggaaggaaaagtagagaaagaaatgccaattccagtccaaagctttgtttgccaagttttcttasaatgaattttaccaatgtatgggttcttgttaacagaatgtgtaacagaaatactgaaagacttttgcctaaagtggcattattgactgctggtgtgatgctactgtaatgcgataaattattaaattgttgcaaagtgaaaaaaaaaaa

SEQ ID NO: 54 - DNA, 23 bp, Artificial Sequence
  Description of Artificial Sequence primer used for generation of a ferritin PCR fragment
ctacgagcgtctcctgaagatgc

SEQ ID NO: 55 - DNA, 32 bp, Artificial Sequence
  Description of Artificial Sequence primer used for generation of a ferritin PCR fragment
cgcggatccaagtcgctgggctcagaaggctc